(19) United States
(12) Patent Application Publication
(10) Pub. No.: US 2025/0087299 A1
(43) Pub. Date: Mar. 13, 2025

(54) IDENTIFICATION OF BINDING SITES IN A PROTEIN STRUCTURE

(71) Applicant: Genentech, Inc., South San Francisco, CA (US)

(72) Inventor: Benjamin Thomas WALTERS, South San Francisco, CA (US)

(21) Appl. No.: 18/960,282

(22) Filed: Nov. 26, 2024

Related U.S. Application Data
(63) Continuation of application No. PCT/US2023/067741, filed on Jun. 1, 2023.
(60) Provisional application No. 63/347,798, filed on Jun. 1, 2022.

(51) Int. Cl. G16B 15/30

(52) U.S. Cl. CPC G16B 15/30

(57) ABSTRACT

In one aspect, a method for identifying residues in a particular conformation of a protein molecule capable of forming a stable binding between a compound and the protein molecule is provided. The method can include identifying, for inclusion in a perturbation set, one or more residues from the conformation of the protein molecule that exhibit a protection factor satisfying one or more thresholds. One or more residues may be excluded from the perturbation set based at least on a spatial arrangement and/or an orientation of the one or more residues. One or more rounds of molecular dynamics simulations whose result is indicative of an interaction between the compound and the residues in the perturbation set may be performed. The residues in the perturbation set may be identified as forming a stable binding between the compound and the protein molecule based at least on the result of the molecular dynamics simulation

### CROSS REFERENCE TO RELATED APPLICATION

This application claims priority to U.S. Provisional Application No. 63/347,798, entitled “IDENTIFICATION OF BINDING SITES IN A PROTEIN STRUCTURE” and filed on Jun. 1, 2022, the disclosure of which is incorporated herein by reference in its entirety.

### TECHNICAL FIELD

The subject matter described herein relates generally to identification of one or more binding sites in conformations of a protein molecule.

### BACKGROUND

Molecular binding is a process in which two or more molecules (e.g., proteins, ligands, etc.) can interact to form a stable molecular structure. For example, molecular binding can include chemical bonding between a portion of a first molecule and a portion of the second molecule. The portion of the first molecule that binds to the second molecule can include multiple binding sites. In the case of a protein-ligand complex, for instance, each binding site in the protein molecule can include multiple binding residues, which are residues participating in the binding with the ligand. Various real-world applications (e.g., drug discovery and/or the like) may require identifying, in one molecules, the binding sites capable of binding with another molecule. For instance, the identities of the binding residues in a protein molecule may be leveraged toward designing ligands that can effectively bind with the protein molecule.

### SUMMARY

Systems, methods, and articles of manufacture, including computer program products, are provided for identifying binding sites in a protein molecule. In some example embodiments, there is provided a system that includes at least one processor and at least one memory. The at least one memory may include program code that provides operations when executed by the at least one processor. The operations may include: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

In another aspect, there is provided a method for identifying binding sites in a protein molecule. The method can include: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

In another aspect, there is provided a computer program product including a non-transitory computer readable medium storing instructions. The instructions may cause operations may executed by at least one data processor. The operations may include: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

In some variations, one or more features disclosed herein including the following features can optionally be included in any feasible combination.

In some variations, the protection factor associated with each residue in the first conformation may correspond to a difference between a first energy of a residue in a bound state and a second energy of the residue in an unbound state.

In some variations, the one or more residues are identified for inclusion in the first perturbation set based at least on the protection factor of the one or more residues satisfying one or more thresholds.

In some variations, the protection factor of the one or more residues may be determined to satisfy the one or more thresholds based at least on a value of the protection factor being within a percentile of a maximum protection factor value observed for the first conformation of the protein molecule.

In some variations, the percentile may be determined based at least on a magnitude of a difference between the maximum protection factor value and a plurality of other protection factor values observed for the first conformation of the protein molecule.

In some variations, the percentile may be a value between 5 and 20.

In some variations, the interaction between the compound and the first plurality of residues in the first perturbation set may include at least a portion of the first plurality of residues in the first perturbation set forming an induced fit pocket within the first conformation of the protein molecule.

In some variations, each round of molecular dynamics simulation may simulate a temporal evolution of a coupled protein-structure-compound including the compound bound to the first conformation of the protein molecule at the first plurality of residues included in the first perturbation set.

In some variations, the coupled protein-structure-compound may be generated based at least on the first perturbation set and a structural information specifying a spatial arrangement of atoms in the first conformation of the protein molecule in an unbound state.

In some variations, the determining the first perturbation set may further include excluding, from the first perturbation set, at least a third residue that requires a threshold quantity of reorientations in order to interact with the compound.

In some variations, the threshold quantity of reorientations may be satisfied by (i) a quantity of atoms in the third residue that require a reorientation in order to interact with the compound, (ii) an angle of reorientation that one or more atoms in the third residue is required to undergo in order to interact with the compound, and/or (iii) a distance of reorientation that the one or more atoms in the third residue is required to undergo in order to interact with the compound.

In some variations, a first vector from a center of geometry (COG) of the first plurality of residues in the first perturbation set and an α-carbon of the third residue may be determined. A second vector from the α-carbon to one of a δ-carbon, γ-carbon, or β-carbon present in the third residue may be determined. The threshold quantity of reorientations may be determined to be satisfied based at least on an angle formed by the first vector and the second vector satisfying one or more thresholds.

In some variations, the third residue may be kept in the first perturbation set based at least on the third residue being glycine.

In some variations, the determining the first perturbation set may further include determining a distance between a first centroid of a first cluster of residues and a second centroid of a second cluster of residues, determining that the distance fails to correspond to one or more dimensions of the compound, and excluding, from the first perturbation set, the first residue based at least on the first residue being a part of the first cluster of residues.

In some variations, the determining the first perturbation set may further include applying, to the first plurality of residues, a clustering technique to partition the first plurality of residues into at least the first cluster of residues and the second cluster of residues.

In some variations, the clustering technique may include one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

In some variations, the determining the first perturbation set may further include determining a first quantity of residues in the first cluster of residues and a second quantity of residues in the second cluster of residues, determining a first mean distance between the first centroid and residues in the first cluster; and determining a second mean distance between the second centroid and residues in the second cluster.

In some variations, the determining the first perturbation set may further include excluding, from the first perturbation set, the first cluster of residues based at least on a determination that (i) the second cluster includes fewer residues than the first cluster, and (ii) the distance between the first centroid of the first cluster and the second centroid of the second cluster exceeds a sum of the first mean distance between the first centroid and residues in the first cluster and the second mean distance between the second centroid and residues in the second cluster.

In some variations, the result may include a distance between a first center of geometry of the compound and a second center of geometry of a plurality of backbone nitrogen (N) atoms in the first plurality of residues in the first perturbation set.

In some variations, the result may include a distance between a first center of geometry of the compound and one or more amide nitrogen atoms present in the first plurality of residues in the first perturbation set.

In some variations, the one or more rounds of molecular dynamics simulation may include a first round of molecular dynamics simulation performed at a first temperature and a second round of molecular dynamics simulation performed at a second temperature.

In some variations, the one or more rounds of molecular dynamics simulation may include a first round of molecular dynamics simulation performed for a first length of time and a second round of molecular dynamics simulation performed for a second length of time.

In some variations, the one or more rounds of molecular dynamics simulation may include a first round of molecular dynamics simulation performed for the first conformation of the protein molecule that is associated with the first perturbation set and a second round of molecular dynamics simulation performed for a second conformation of the protein molecule that is associated with a second perturbation set.

In some variations, each of the first conformation and the second conformation may be a three-dimensional structure having a different spatial arrangement of atoms in the protein molecule.

In some variations, the second perturbation set for the second conformation of the protein molecule may be determined. The determining may include at least one of (i) identifying, based at least on the protection factor of at least a portion of residues in the protein molecule, one or more residues for inclusion in the second perturbation set, (ii) excluding, from the second perturbation set, at least a third residue whose distance to a fourth residue in the second perturbation set fails to correspond to the one or more dimensions of the compound, and (iii) excluding, from the second perturbation set, at least a fifth residue that requires a threshold quantity of reorientations in order to interact with the compound.

In some variations, the one or more rounds of molecular dynamics simulation may include a plurality of rounds of molecular dynamics simulation. Each round of molecular dynamics simulation may subject a coupled compound-protein-structure including the compound bound to the protein molecule at the first plurality of residues in the first perturbation set to a different condition.

In some variations, the result of the one or more rounds of molecular dynamics simulation may include one or more conditions in which the coupled compound-protein-structure disassociates.

In some variations, the one or more conditions may include a temperature at which the coupled compound-protein-structure dissociates.

In some variations, the one or more conditions may include a length of time after which the coupled compound protein-structure dissociates.

In some variations, the one or more rounds of molecular dynamics simulation may include a round of molecular dynamics simulation that is performed at a higher temperature than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

In some variations, the one or more rounds of molecular dynamics simulation may include a round of molecular dynamics simulation that is performed at a lower temperature than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

In some variations, the one or more rounds of molecular dynamics simulation may include a round of molecular dynamics simulation that is performed for a longer time period than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

In some variations, the one or more rounds of molecular dynamics simulation may include a round of molecular dynamics simulation that is performed for a shorter time period than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

In some variations, the first plurality of residues in the perturbation set may be identified as forming a stable binding by at least determining, based at least on the result of the one or more rounds of molecular dynamics simulations, one or more metrics quantifying a binding affinity between the compound and the first conformation of the protein molecule, and determining, based at least on the one or more metrics, the first plurality of residues in the perturbation set as forming the stable binding between the compound and the first conformation of the protein molecule.

In some variations, the determining the first perturbation set may further include determining a distance between the first residue and the second residue, and in response to determining that the distance between the first residue and the second residue exceeds the one or more dimensions of the compound, excluding the first residue from the first perturbation set.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to identifying binding sites in a protein molecule, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the subject matter disclosed herein. The patent or application file contains at least one drawing executed in color. Copies of this patent or patent application publication with color drawings will be provided by the Office upon request and payment of the necessary fee. In the drawings,

FIG. 1 depicts a system diagram illustrating an example of a protein structure analysis system 100, in accordance with some example embodiments;

FIG. 2A depicts a flowchart illustrating an example of a process for identifying residues in a protein molecule capable of providing a stable binding between a compound and a protein molecule, in accordance with some example embodiments;

FIG. 2B depicts a flowchart illustrating an example of a process for determining one or more bound conformations of a protein molecule bound to a compound, in accordance with some example embodiments;

FIG. 3 depicts a flowchart illustrating an example of a process for determining a perturbation set, in accordance with some example embodiments;

FIG. 4 depicts an example of a graph illustrating the protection factors of the residues included in the protein molecule, in accordance with some example embodiments;

FIG. 5 depicts an example of a compound and the residues in a protein molecule identified as binding sites between the compound the protein molecule, in accordance with some example embodiments;

FIG. 6 depicts a schematic diagram illustrating an example of a protein molecule and the clusters of residues identified within the protein molecule, in accordance with some example embodiments;

FIG. 7 depicts a schematic diagram illustrating the displacement between the respective centers of geometry of two clusters of residues within a protein molecule, in accordance with some example embodiments;

FIG. 8 depicts a schematic diagram illustrating the exclusion of certain residues from the initial perturbation set of a protein molecule, in accordance with some example embodiments;

FIG. 9 depicts a schematic diagram illustrating a portion of an example of a protein molecule having at least two sidechains that are oriented in different directions, in accordance with some example embodiments;

FIG. 10 depicts a schematic diagram illustrating the exclusion of certain residues from a perturbation set based on the orientation of the residues, in accordance with some example embodiments;

FIG. 11 depicts a block diagram illustrating an exemplary schematic of an analysis controller, in accordance with some example embodiments;

FIG. 12 depicts a system diagram illustrating an exemplary implementation of an analysis controller, in accordance with some example embodiments;

FIG. 13 depicts a system diagram illustrating an example of a process for identifying a perturbation set, in accordance with some example embodiments; and

FIG. 14 depicts a block diagram illustrating an example of a computing system, in accordance with some example embodiments.

When practical, similar reference numbers are used to refer to same or similar items in the drawings.

### DETAILED DESCRIPTION

The interaction between two molecules, such as the binding between a compound (e.g., a ligand and/or the) and a protein molecule, can play a significant role in biological processes. Thus, understanding the dynamics of protein-compound interaction(s), including the identification of potential binding sites on a protein molecule for binding with a compound, can be desirable for various scientific endeavors (e.g., drug discovery). The binding affinity between a protein molecule and a compound may depend whether the three-dimensional structure (e.g., the secondary structure, the tertiary structure, and/or the like) of the protein molecule complements that of the compound. However, due to its flexible nature, the three-dimensional structure of a protein molecule may evolve over time, particularly as the protein molecule comes into close proximity to and interacts with the compound. Furthermore, in some cases, a population of protein molecules having the same primary structure (e.g., sequence of amino acid residues) may still exhibit different three-dimensional structures. The terms “protein conformation,” “conformation,” and “conformer” may be used interchangeably herein to refer to a specific three-dimensional structure of a protein molecule. The same sequence of amino acid residues may be associated with a conformational ensemble (CE) containing multiple conformations (conformers), each of which being defined by the spatial arrangement of the atoms in the constituent amino acid residues.

Existing techniques capable of identifying portions of a protein molecule that are energetically favorable for binding with the compound ignore the dynamic nature of the protein molecule during the binding process including the changes in the three-dimensional structure of the protein molecule that arise when the protein molecule comes into close proximity to and interacts with the compound. Consequently, conventional techniques are inaccurate in instances where the structural dynamics of the protein molecule influence the binding between the protein molecule and the compound. Other existing techniques rely on trial-and-error where the binding energies between the compound and the protein molecule are calculated at multiple locations on a specific conformation of the protein molecule before a location with minimum binding energy is identified. However, a purely trial-and-error based approach, which consume excessive computational resources and impose extensive runtimes, is too inefficient to be practicable.

Interaction between the compound and the protein molecule (e.g., during the binding process) can alter the three-dimensional structure of the protein molecule. For example, the presence of the compound can cause a reorientation of one or more amino acid residues in the protein molecule, thus affecting its three-dimensional structure. Such changes can affect the ability of at least some residues for forming a stable binding between the compound and the protein molecule. For instance, one or more residues in the protein molecule may reorient to form an induced-fit pocket, in which case the residues forming the pocket will have a higher likelihood of forming a stable binding with the compound. Nevertheless, as noted, existing techniques for identifying binding sites in the protein molecule disregard the dynamic nature of the protein molecule, including those engendered by the interaction between the protein molecule and the compound.

By contrast, in accordance with some implementations of the current subject matter, an analysis controller may identify a perturbation set, which includes one or more residues in a particular conformation of the protein molecule that are capable of forming a stable binding with the compound. The analysis controller may identify the perturbation set for each conformation of the protein molecule in an iterative manner in order to account for the structural changes that the protein molecule undergoes when interacting and binding with the compound. Moreover, it should be appreciated that the perturbation set of a protein conformation does not necessarily include every residue that will interact and bind with the compound. That is, while the residues included the perturbation set are more likely to interact with the compound to form a bond between the compound and the corresponding protein conformation, other residues in the protein molecule that are not identified for inclusion in the perturbation set may still interact with the compound as a part of the binding process.

In some example embodiments, the analysis controller may generate, for each conformation of a protein molecule, a separate perturbation set. For example, the analysis controller may identify, for each conformation of the protein molecule, one or more residues in that conformation for inclusion in a perturbation set. In some cases, the one or more residues may be identified for inclusion in the perturbation set of a protein conformation based on the protection factor associated with the individual residue in that protein conformation. Moreover, in some cases, the perturbation set for the protein conformation may be further updated to exclude one or more residues based on the geometric characteristics of the residues including, for example, the relative location, the orientation, and/or the like of the residues in that particular conformation of the protein molecule. The analysis controller may determine the structural information (e.g., three-dimensional coordinates and/or the like) of a coupled protein-structure-compound formed by the compound bound to the conformation of the protein molecule at the residues included in the perturbation set before performing one or more rounds molecular dynamic (MD) simulations on the coupled protein-structure-compound. For example, each round of molecular dynamics simulation may include simulated movements to dissociate the coupled protein-structure-compound at a different temperature. In some cases, a round of molecular dynamics simulation may be performed at a different temperature than another round (e.g., a previous round) of molecular dynamics simulation. Alternatively, and/or additionally, a round of molecular dynamics simulation may be performed at a same temperature as another round (e.g., a previous round) of molecular dynamics simulation but for an additional time period. The analysis controller may determine, based at least on the results of the molecular dynamics simulations, whether the residues in the perturbation set form a stable binding between the protein conformation and the compound.

As noted, in some example embodiments, one or more residues in a particular protein conformation may be selected for inclusion in a corresponding perturbation set based at least on the protection factor associated with the individual residues in the protein conformation. The protection factor of a residue may be indicative of the ability of the residue to interact with the compound, for example, to bind with the compound. For example, in some cases, the protection factor of the residue may correspond to a difference between a first energy of the residue in a bound state and a second energy of the residue in an unbound state. In some cases, the protection factor of the residue may be determined based on the amide hydrogen exchange (HX) rates of the protein conformation, the values for which can be determined experimentally, for example, through mass spectroscopy, nuclear magnetic resonance, and/or the like. A residue exhibiting a higher protection factor has a higher a priori likelihood of binding with the compound than a residue exhibiting a lower protection factor. Accordingly, the analysis controller may identify, for inclusion in the perturbation set, one or more residues in the protein molecule if the protection factor of the one or more residues satisfy one or more thresholds. In doing so, the analysis controller may identify, for inclusion in the perturbation set, one or more residues in the conformation of the protein molecule whose likelihood of forming a stable binding with the compound satisfy one or more thresholds.

In some cases, the ability of two separate residues in a particular conformation of the protein molecule to simultaneously bind with the compound may depend on their spatial arrangement, such as their relative locations, in that conformation the protein molecule. For example, if the distance (e.g., Euclidean distance and/or the like) between a first residue and a second residue exceeds one or more dimensions of the compound (e.g., greater than the largest dimension associated with the compound), at least one of the residues may be excluded from the perturbation set at least because the compound is unable to simultaneously bind to both the first residue and the second residue. Accordingly, in some cases, the analysis controller may determine the perturbation set of a protein conformation by at least excluding one or more residues based on a location of the residue in that conformation of the protein molecule. For instance, upon determining that the compound is unable to simultaneously bind to both the first residue and the second residue, the analysis controller may include the first residue in the perturbation set but not the second residue. As described in more detail below, in some cases, the analysis controller may further refine the perturbation set with the first residue by at least excluding one or more residues whose orientation prevents them from forming a stable binding with the compound. Moreover, it should be appreciated that the perturbation set of a particular protein conformation may be rebuilt with one or more changes to its constituent residues based on the results of molecular dynamics simulation (MD). For example, the perturbation set may be rebuilt in cases where the protein molecule and the compound dissociates after one or more rounds of molecular dynamics simulation (MD) before one or more additional rounds of molecular dynamics simulation (MD) are performed based on the rebuilt perturbation set.

In some cases, two or more clusters of residues may be incapable of simultaneously binding with the compound due to the spatial arrangement of the two or more clusters of residues within a particular protein conformation. For example, if the distance (e.g., Euclidean distance and/or the like) between a first cluster of residues and a second cluster of residues exceeds one or more dimensions of the compound (e.g., greater than the largest dimension of the compound), at least one cluster of residues may be excluded from the perturbation set because the compound cannot bind with both clusters simultaneously. In some cases, the analysis controller may identify one or more clusters of residues based on the respective positions (e.g., the three-dimensional coordinates and/or the like) of individual residues in the protein conformation. Moreover, in some cases, the analysis controller may identify the one or more clusters of residues by at least applying a clustering technique including, for example, k-means clustering, mean-shift clustering, density-based spatial clustering of applications with noise (DBSCAN), expectation-maximization (EM) clustering using Gaussian mixture models (GMM), an agglomerative hierarchical clustering, and/or the like. As described in more detail below, in some cases, the analysis controller may further refine the perturbation set with the first cluster of residues by at least excluding one or more residues whose orientation prevents them from forming a stable binding with the compound. Moreover, in some cases, the perturbation set for the conformation of the protein molecule may be rebuilt if the protein molecule and the compound dissociates after one or more rounds of molecular dynamics simulation (MD) such that one or more additional rounds of molecular dynamics simulation (MD) may be performed based on the rebuilt perturbation set.

As noted, in some example embodiments, the analysis controller may determine the perturbation set by at least excluding, from the perturbation set, one or more residues based at least on the orientation of the residue in a particular conformation of the protein molecule. For example, in some cases, the analysis controller may exclude a residue at least because the orientation of the residue reduces the likelihood of binding between the residue and the compound. The exclusion of the residue from the perturbation set corresponds to the observation that a binding between the residue and the compound can have a higher energy threshold in instances where the orientation of the residue relative to the compound (e.g., when the compound approaches the protein molecule) requires the residue to undergo significant reorientation in order to bind with the compound. Accordingly, in some cases, the analysis controller may exclude the residue if the orientation of the residue within the protein conformation requires the residue to undergo a threshold quantity of reorientations in order to bind with the compound. In some cases, this threshold quantity of reorientations may be satisfied, for example, by the reorientation of a threshold quantity of atoms in the residue (e.g., a threshold quantity of sidechain atoms and/or backbone atoms). Alternatively, and/or additionally, this threshold quantity of reorientations may be satisfied by the angle and/or the distance of the reorientation of one or more atoms in the residue.

In some example embodiments, the analysis controller may determine the stability of the binding provided by the residues in the perturbation set, which are identified based at least on the protection factor, the spatial arrangements, and/or the orientation of individual residues (or clusters of residues) within the corresponding conformation of the protein molecule. For example, in some cases, the stability of the binding provided by the residues in the perturbation set may be determined by at least performing one or more rounds of molecular dynamics simulations (MD) based on a coupled protein-structure-compound in which the compound is bound to the conformation of the protein molecule at the residues included in the perturbation set. The coupled protein-structure-compound may be determined by applying one or more docking algorithms to generate, based on the perturbation set and the unbound structural information of the protein conformation, the complex structural information of the coupled protein-structure-compound. In some cases, molecular dynamics simulations may be performed based on the complex structural information of the coupled protein-structure-compound as set forth, for instance, in a complex coordinate file associated with the coupled protein-structure-compound.

In some example embodiments, the analysis controller may perform iterative rounds of the molecular dynamics simulation (MD) in which each round of the molecular dynamics simulation subjects the coupled protein-structure-compound to different conditions until, for example, the coupled protein-structure-compound dissociates. For example, in some cases, a first round of molecular dynamics simulation may be performed at a different temperature that a second round of molecular dynamics simulation in order to simulate the time evolution of the coupled protein-structure-compound at multiple temperatures. Alternatively, and/or additionally, the second round of molecular dynamics simulation may be performed at a same temperature as the first round of molecular dynamics simulation but for an additional time period. In some cases, if the coupled protein-structure-compound does not dissociate after one or more rounds of the molecular dynamics simulation performed at a first temperature, the analysis controller may perform one or more additional rounds of molecular dynamics simulation at a second temperature that may be, in some cases, higher than the first temperature. The analysis controller may continue to perform additional arounds of molecular dynamics simulation on the coupled protein-structure compound at successively higher temperatures and/or for longer time periods until the coupled protein-structure-compound dissociates. In doing so, the analysis controller may determine and/or rank the stability of the bond formed by the residues in the perturbation set by at least determining the temperature at which the coupled protein-structure-compound dissociates and/or the time duration of the coupled protein-structure-compound (e.g., a length of time after which the coupled protein-structure-compound dissociates). For instance, where the coupled protein-structure-compound is determined to dissociate at a higher temperature and/or after a longer time period, the analysis controller may determine a greater stability in the binding between the compound and the protein conformation at the residues included in the perturbation set.

In some example embodiments, the analysis controller may perform multiple rounds of molecular dynamics simulation (MD) for different conformations of the protein molecule in order to account for variations in the three-dimensional structure of the same protein molecule, which are observed over time as well as in a population of the same molecule due to the folding of the protein molecule. For example, in some cases, the analysis controller may perform a first round of molecular dynamics simulation for a first conformation of the protein molecule and a second of molecular dynamics simulation for a second conformer of the protein molecule. Here, the analysis controller may receive, for each conformation of the protein molecule, structural information (e.g., an unbound coordinate file) indicating, for example, the spatial arrangement of various atoms in the protein molecule (e.g., the spatial location of the individual atoms relative to one another). Moreover, each conformation of the protein molecule may be associated with a different perturbation set at least because a change in the spatial three-dimensional structure of the protein molecule can effect a corresponding change in the residues that are more likely to form a stable binding with the compound. Accordingly, the analysis controller may generate a first perturbation set for the first conformation of the protein molecule and a second perturbation set for the second conformation of the protein molecule. Each of the first perturbation set and the second perturbation set may include residues identified, for example, based on the protection factors, the spatial arrangements, and/or the orientation of the individual residues (or clusters of residues) within the corresponding conformation of the protein molecule. The analysis controller may determine, based at least on the results of the molecular dynamics simulation (MD), a relative binding affinity and/or rank between the compound and the protein molecule as indicated by the stability the bond between the compound and the protein molecule across multiple conformations of the protein molecule.

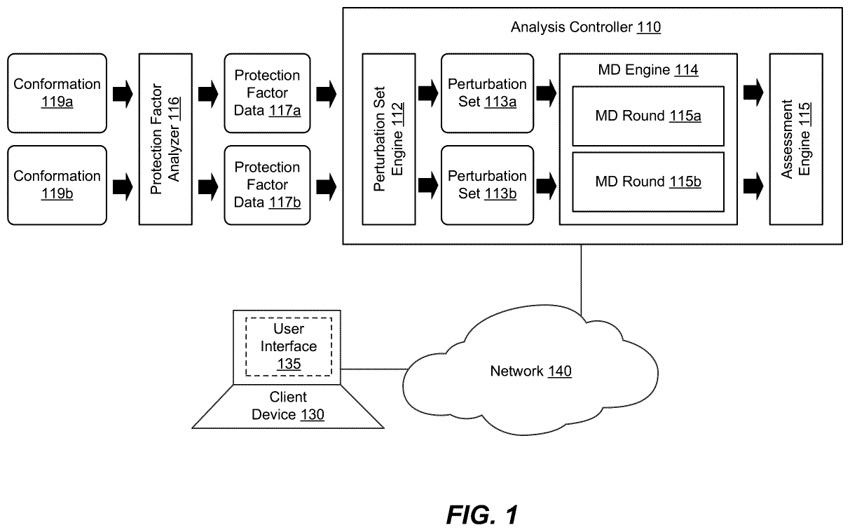

FIG. 1 depicts a system diagram illustrating an example of a protein structure analysis system 100, in accordance with some example embodiments. Referring to FIG. 1, the protein structure analysis system 100 may include an analysis controller 110 that is communicatively coupled with a client device 130 via a network 140. The network 140 may be a wired network and/or a wireless network including, for example, a local area network (LAN), a virtual local area network (VLAN), a wide area network (WAN), a public land mobile network (PLMN), the Internet, and/or the like. The client device 130 may be a processor-based device including, for example, a workstation, a desktop computer, a laptop computer, a high-performance computer, a smartphone, a tablet computer, a wearable apparatus, and/or the like.

In some example embodiments, the analysis controller 110 may identify one or more residues in a protein molecule capable of forming a stable binding with a compound (e.g., a ligand and/or the like). As shown in FIG. 1, in some cases, the analysis controller 110 may include a perturbation set engine 112 that identifies, for inclusion in each perturbation set associated with the protein molecule, one or more residues capable of forming a stable bond between a corresponding conformation of the protein molecule and the compound. For example, in some cases, the one or more residues capable of providing a stable bond with the compound may be identified based on the protection factor, the spatial arrangement, and/or the orientation of the individual residues in the corresponding conformation of the protein molecule. Moreover, in the example shown in FIG. 1, the analysis controller 110 may include a molecular dynamics simulation (MD) engine 114 that performs one or more rounds of molecular dynamics simulation.

In some cases, the molecular dynamics simulation engine 114 may perform one or more rounds of molecular dynamics simulation for each perturbation set, with each round of molecular dynamics simulation being performed at a different temperature or, in some cases, at a same temperature but for an additional time period. For example, the molecular dynamics simulation engine 114 may perform, for a first perturbation set 113a of a first conformation 119a of the protein molecule, a first round of molecular dynamics simulation 115a at a first temperature and a second round of molecular dynamics simulation 115b at either the same first temperature or a different second temperature. As shown in FIG. 1, the analysis controller 110 may include an assessment engine 118 that determines, based at least on the results of the first round of molecular dynamics simulation 115a and the second round of molecular dynamics simulation 115b, if the residues included in the first perturbation set 113a form a stable binding between the compound and the first conformation 119a of the protein molecule.

As described in more detail below, in instances where the perturbation set engine 112 generates multiple perturbation sets, including a separate perturbation set for each conformation of the protein molecule, the molecular dynamics simulation engine 114 may perform one or more rounds of molecular dynamics simulation for each perturbation set (e.g., at different temperatures, for different lengths of time, and/or the like). For example, the molecular dynamics simulation engine 114 may perform the first round of molecular dynamics simulation 115a for the first perturbation set 113a of the first conformation 119a and the second round of molecular dynamics simulation 115b for a second perturbation set 113b of a second conformation 119b. In these instances, the assessment engine 118 may further determine, based at least on the results of the first round of molecular dynamics simulation 115a and the second round of molecular dynamics simulation 115b, the stability of the bond between the protein molecule and the compound across, for example, multiple conformations of the protein molecule.

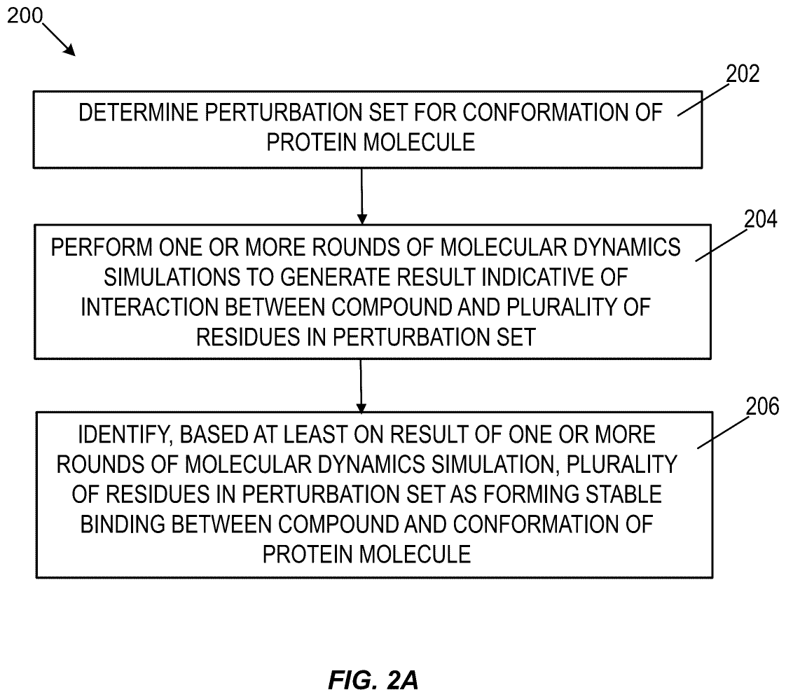

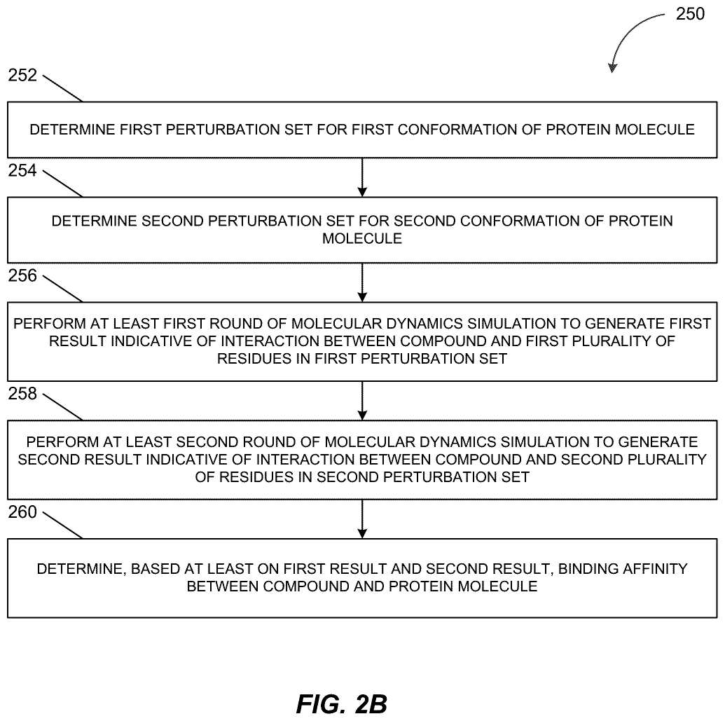

To further illustrate, FIG. 2A depicts a flowchart illustrating an example of a process 200 for identifying residues in a particular conformation of a protein molecule capable of forming a stable binding between a compound and the protein molecule, in accordance with some example embodiments. Referring to FIGS. 1 and 2A, the process 200 can be performed by the analysis controller 110 to determine, for example, if the residues included in the perturbation set of a protein conformation form a stable binding between the protein molecule and a compound (e.g., a ligand and/or the like).

At 202, the analysis controller 110 may determine a perturbation set for a conformation of a protein molecule. In some example embodiments, the analysis controller 110, for example, the perturbation set engine 112, may generate the first perturbation set 113a to include one or more residues from the first conformation 119a of a protein molecule. In some cases, the perturbation set engine 112 may generate the first perturbation set 113a by at least identifying, for inclusion in the first perturbation set 113a, one or more residues based at least on the protection factor, the spatial arrangement, and/or the orientation of the individual residues in the first conformation 119a of the protein molecule. For example, in some cases, the perturbation set engine 112 may determine to include, in the first perturbation set 113a, a first residue based at least on the protection factor of the first residue satisfying one or more thresholds. In the example shown in FIG. 1, the protection factor of the first residue may be included in a first protection factor data 117a received from a protection factor analyzer 116 (e.g., a mass spectrometer, a nuclear magnetic resonance (NMR) device, and/or the like). In some cases, the protection factor of the first residue may satisfy the one or more thresholds if its value falls within a certain percentile (or quantile) of the protection factor values observed in the first conformation 119a of the protein molecule. For instance, in some cases, the protection factor of the first residue may satisfy the one or more thresholds if its value is within X %, wherein X may be a value between 5 and 20, of the maximum protection factor value (or the maximum log (PF)). In that case, if the maximum log (PF) is 2, then the protection factor of the first residue will fall into the 20 percentile if its value is greater than 1.6. Alternatively, if the maximum log (PF) is 4, then the protection factor of the first residue will fall into the 20 percentile if its value is greater than 3.2. It should be appreciated that the value of the percentile X may be determined and, in some cases, adjusted in accordance with the magnitude of the difference between the maximum protection factor value observed for the first conformation 119a of the protein molecule and the other protection factor values of other residues in the first conformation 119a of the protein molecule.

However, in some cases, the perturbation set engine 112 may subsequently determine to exclude the first residue from the first perturbation set 113a if the perturbation engine 112 determines that the distance (e.g., the Euclidean distance) between the first residue (or a first cluster of residues including the first residue) and a second residue (or a second cluster of residues including the second residue) in the first conformation 119a of the protein molecule exceeds one or more dimensions (e.g., the largest dimension) associated with the compound. As will be described in more detail below, residues in the first conformation 119a of the protein molecule can be grouped into one or more clusters and, in some cases, the first cluster of residues may be excluded from the first perturbation set 113a if the distance (e.g., the Euclidean distance and/or the like) between, for example, the first centroid of the first cluster and the second centroid of the second cluster exceeds one or more dimensions of the compound such that the compound is unable to bind simultaneously to both the residues in the first cluster as well as the residues in the second cluster. The exclusion of the residues in the first cluster reflects the observation that the compound bound to the residues in the second cluster is unable to also bind to the residues in the first cluster at the same time.

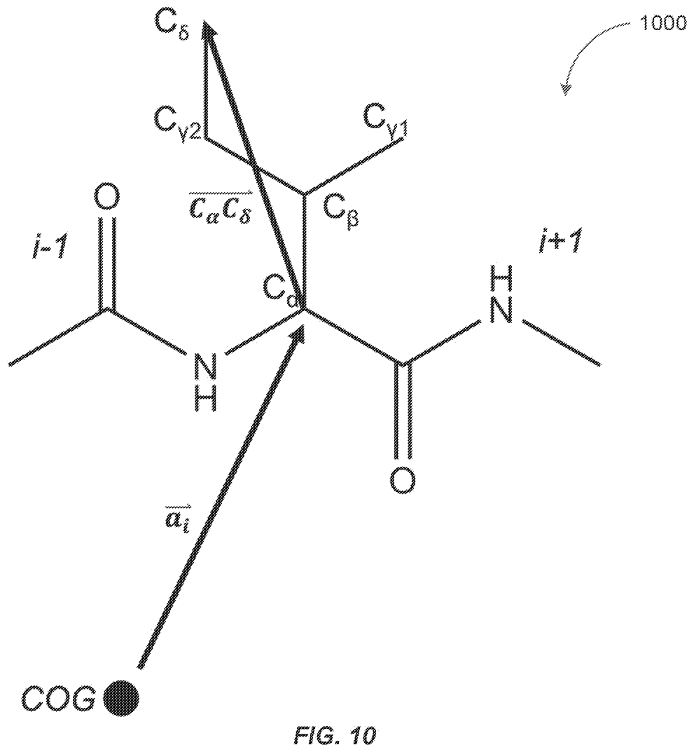

In some cases, the perturbation set engine 112 may further determine the first perturbation set 113a by at least excluding, from the first perturbation set 113a, one or more residues based at least on the orientation of the one or more residues. As will be described in more detail with respect to FIG. 10, the orientation of a residue may be determined based on the location of one or more of an α-carbon (c_{α}) in the residue, a center of geometry (COG) of the perturbation set containing the residue, and a β-carbon (c_{β}), δ-carbon (c_{δ}), or γ-carbon (c_{γ}) in the residue. In some cases, the residue may be excluded from the first perturbation set 113a if the residue is required to undergo a threshold quantity of reorientations in order to bind with the compound. For example, in some cases, this threshold quantity of reorientations may be satisfied if the quantity of atoms in the residue required to undergo reorientation satisfies one or more thresholds. Alternatively, and/or additionally, the threshold quantity of reorientations may be satisfied if the angle and/or the distance of the reorientation of one or more atoms in the residue satisfy one or more thresholds. In some cases, the residue may be excluded from the first perturbation set 113a if a sidechain of the residue is required to undergo the threshold quantity of reorientations in order to bind with the compound. In these instances, the threshold quantity of reorientations may be satisfied if the angle between a first vector (defined by a first location of the center of geometry of the perturbation set containing the residue and a second location of the α-carbon (c_{α})) and a second vector (defined by the second location of the α-carbon (c_{α}) and a third location of the β-carbon (c_{β}), δ-carbon (c_{δ}), or γ-carbon (c_{γ})) satisfies one or more thresholds.

At 204, the analysis controller 110 may perform one or more rounds of molecular dynamics simulations to generate a result indicative of an interaction between the compound and the plurality of residues in the perturbation set. In some example embodiments, the molecular dynamics simulation (MD) engine 114 may perform one or more rounds of molecular dynamics simulation (MD) based at least on the first perturbation set 113 generated, for example, at operation 202 and structural information describing the spatial arrangement of atoms within the first conformation 119a of the protein molecule. For example, in some cases, the structural information associated with the first conformation 119a of the protein molecule may indicate the locations (e.g., coordinates) of atoms in the protein molecule. In some cases, in addition to the structural information of the first conformation 119a of the protein molecule, the molecular dynamic simulation engine 114 can also receive additional information such as the position of one or more heavy atoms (e.g., oxygen, nitrogen, carbon, and/or) in the first conformation 119a of the protein molecule, experimental pH used to define the ionization states on sidechains of the protein molecule, force field generated for the compound (e.g., ligand), and/or the like.

In some example embodiments, the analysis controller 110 may generate, based at least on the first perturbation set 113a and the structural information of the first conformation 119a of the protein molecule, a coupled compound-protein-structure (e.g., a protein-ligand complex). For example, in some cases, the analysis controller 110 may apply a docking algorithm to generate the coupled compound-protein-structure where the compound is bound to the protein molecule at the residues included in the first perturbation set. Moreover, in some cases, the analysis controller 110 may generate, based at least on the first perturbation set 113a and the unbound structural information of the first conformation 119a of the protein molecule, the complex structural information of the coupled compound-protein-structure. In some cases, the molecular dynamics simulation engine 114 may perform, based at least the complex structural information of the coupled compound-protein-structure, one or more rounds of molecular dynamics simulation. For instance, the molecular dynamics simulation engine 114 may perform the first round of molecular dynamics simulation 115a at a first temperature and the second round of molecular dynamics simulation 115b at the same first temperature for an additional length of time or at a second temperature (that is different than the first temperature). The iterative rounds molecular dynamic simulation (MD) are described in further detail in FIGS. 2B, 11, and 12.

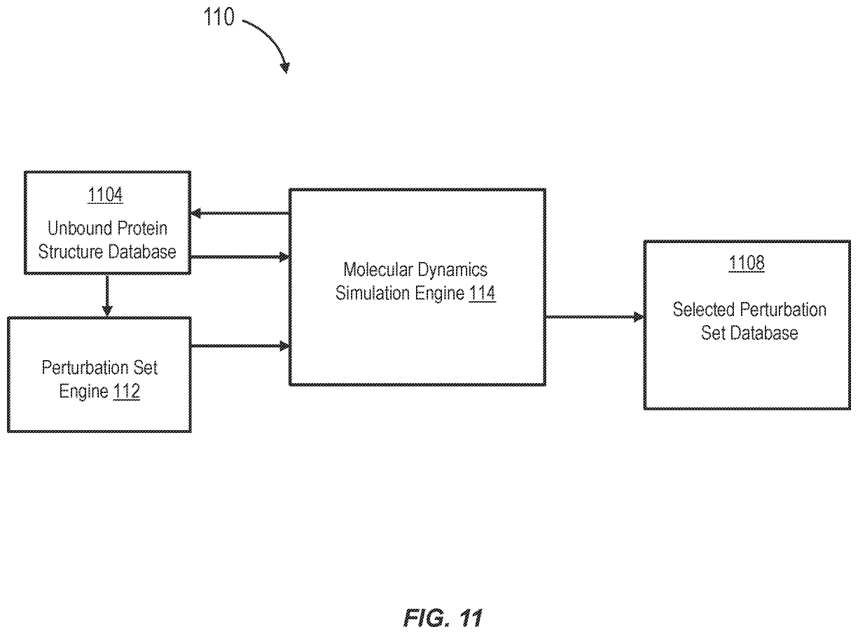

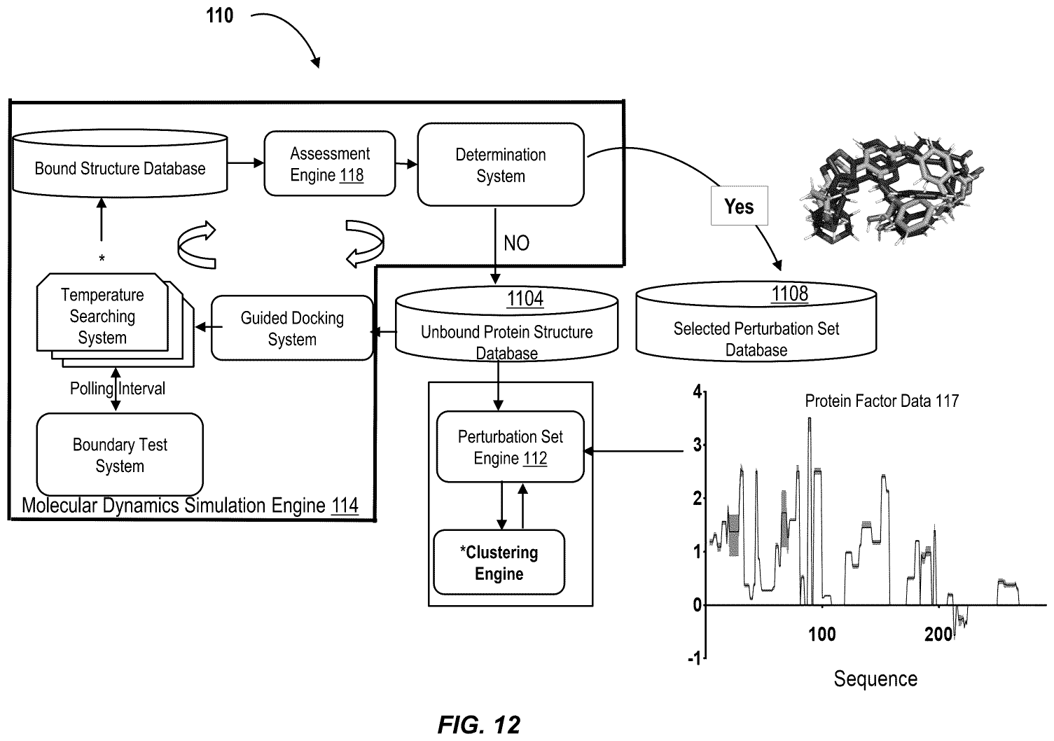

At 206, the analysis controller 110 may identify, based at least on the result of the one or more rounds of molecular dynamics simulation, the plurality of residues in the perturbation set as forming a stable binding between the compound and the conformation of the protein molecule. In some example embodiments, the results of the one or more rounds of molecular dynamics simulation, such as that from the first round of molecular dynamics simulation 113a and the second round of molecular dynamics simulation 113b, may include a temperature at which the coupled compound-protein-structure dissociated and/or a time duration of the coupled compound-protein-structure (e.g., how long the coupled compound-protein-structure remained bound). In some cases, the assessment engine 118 may determine, based at least on the results of the molecular dynamics simulations, one or more metrics quantifying the binding affinity between the compound and the first conformation 119a of the protein molecule (e.g., a satisfaction score, a divergence score, and/or the like). Moreover, in some cases, the assessment engine 118 may identify, based at least on the one or more metrics satisfying one or more thresholds, the residues in the perturbation set as forming a stable binding between the compound and the first conformation 119a of the protein molecule. The analysis performed to identify a perturbation set as forming a stable binding between a compound and a particular conformation of a protein molecule is described in further detail in FIGS. 11 and 12.

FIG. 2B depicts a flowchart illustrating another example of a process 250 for determining one or more bound conformations of a protein molecule bound to a compound, in accordance with some example embodiments. Referring to FIGS. 1 and 2B, the process 250 can be performed by the analysis controller 110 to determine, based on the binding stability between the compound and multiple conformations of the protein molecule, the binding affinity between the protein molecule and a compound (e.g., a ligand and/or the like). As noted, the folding of the protein molecule may give rise to changes in the three-dimensional structure of the protein molecule. For example, the protein molecule may assume the first conformation 119a at a first timepoint and the second conformation 119b at a second timepoint. Moreover, at any point in time, a population of the same protein molecule may include multiple conformations of the protein molecule including, for example, the first conformation 119a and the second conformation 119b. Changes in the conformation of the protein molecule may change which residue in the protein molecule that are capable of forming a stable binding with the compound. Accordingly, in some example embodiments, the analysis controller 110 may determine multiple perturbation sets, each of which being associated with a different conformation of the protein molecule and including one or more residues in the protein molecule capable of forming a stable bond with the compound while the protein molecule assumes the corresponding three-dimensional structure. As described in more detail below, the process 250 may be performed to account for the structural dynamics of the protein molecule, including the time evolution of its three-dimensional structure and the concomitant variations in the interaction between the protein molecule and the compound.

At 252, the analysis controller 110 may determine a first perturbation set for a first conformation of a protein molecule. In some example embodiments, the analysis controller 110, for example, the perturbation set engine 112, may determine the first perturbation set 113a for the first conformation of the protein molecule. In the example shown in FIG. 1, the perturbation set engine 112 may determine the first perturbation set 113a based at least on the first protection factor data 117a (e.g., received from the protection factor analyzer 116) associated with the first conformation 119a of the protein molecule. Alternatively, and/or additionally, the perturbation set engine 112 may determine the first perturbation set 113a based on a spatial arrangement and/or an orientation of the atoms in the individual residues forming the first conformation 119a of the protein molecule.

At 254, the analysis controller 110 may determine a second perturbation set for a second conformation of the protein molecule. In some example embodiments, the analysis controller 110, for example, the perturbation set engine 112, may determine the second perturbation set 113b for the second conformation of the protein molecule. For instance, in the example shown in FIG. 1, the perturbation set engine 112 may determine the second perturbation set 113b based at least on a second protection factor data 117b (e.g., received from the protection factor analyzer 116) associated with the second conformation 119b of the protein molecule. Alternatively, and/or additionally, the perturbation set engine 112 may determine the second perturbation set 113b based on a spatial arrangement and/or an orientation of the atoms in the individual residues forming the second conformation 119b of the protein molecule.

At 256, the analysis controller 110 may perform at least a first round of molecular dynamics simulation to generate a first result indicative of an interaction between a compound and a first plurality of residues in the first perturbation set. In some example embodiments, the analysis controller 110, for example, the molecular dynamics simulation engine 114 may perform one or more rounds of molecular dynamics simulation (MD), such as the first round of molecular dynamics simulation 115a, on a first coupled compound-protein-structure corresponding to the compound bound to the first conformation 119a of the protein molecule at the residues included in the first perturbation set 113a. In some cases, the molecular dynamics analysis engine 114 may perform multiple rounds of molecular dynamics simulation on the first coupled compound-protein-structure, for example, at different temperatures, for different time durations, and/or the like. Multiple rounds of molecular dynamics simulation may be performed to determine, for example, a first temperature at which the first coupled compound-protein-structure dissociates and/or a first time duration of the first coupled compound-protein-structure. In doing so, the analysis controller 110, for example, the assessment engine 118 may determine if the first conformation 119a of the protein molecule is able to form a stable binding with the compound at the residues included in the first perturbation set 113a.

At 258, the analysis controller 110 may perform at least a second round of molecular dynamics simulation to generate a second result indicative of an interaction between the compound and a second plurality of residues in the second perturbation set. In some example embodiments, the analysis controller 110, for example, the molecular dynamics simulation engine 114 may perform one or more rounds of molecular dynamics simulation (MD), such as the second round of molecular dynamics simulation 115b, on a second coupled compound-protein-structure corresponding to the compound bound to the second conformation 119b of the protein molecule at the residues included in the second perturbation set 113b. For example, in some cases, the molecular dynamics analysis engine 114 may perform multiple rounds of molecular dynamics simulation on the second coupled compound-protein-structure at different temperatures, for different time durations, and/or the like. Moreover, in some cases, multiple rounds of molecular dynamics simulation may be performed to determine, for example, a second temperature at which the second coupled compound-protein-structure dissociates and/or a second time duration of the second coupled compound-protein-structure. In doing so, the analysis controller 110, for example, the assessment engine 118, may determine if the second conformation 119b of the protein molecule is able to for a stable binding with the compound at the residues included in the second perturbation set 113b.

At 260, the analysis controller 110 may determine, based at least on the first result and the second result, a binding affinity between the compound and the protein molecule. In some example embodiments, the analysis controller 110, for example, the assessment engine 118 may determine, based at least on the results of the molecular dynamics simulation performed on different conformations of the protein molecule, the binding affinity between the compound and multiple conformations of the protein molecule. As noted, the assessment engine 118 may determine the stability of the bond between the compound and the protein molecule in the first conformation 119a (e.g., at operation 256) and the stability of the bond between the compound and the protein molecule in the second conformation 119b (e.g., at operation 258). Doing so may account for the structural dynamics of the protein molecule, which includes changes in its three-dimensional structure over time.

Accordingly, in some cases, the assessment engine 118 may further determine, based on the stability of the bond between the compound and various conformations of the protein molecule, one or more metrics quantifying the binding affinity between the compound and the protein molecule (e.g., a satisfaction score, a divergence score, and/or the like). For example, in some cases, the assessment engine 118 may determine that the binding affinity between the compound and the protein molecule satisfy one or more thresholds if the compound is able to form a stable bond with a threshold quantity of conformations of the protein molecule. Alternatively, and/or additionally, the assessment engine 118 may identify, based at least on the results of the first round of molecular dynamics simulation 115a performed on the first conformation 119a and the second round of molecular dynamics simulation 115b performed on the second conformation 119b, one or more of the first conformation 119a and the second conformation 119b as a stable docked conformation (or most stable conformation) of the protein molecule. In cases where the compound is able to form stable bindings with multiple conformations of the protein molecule, the assessment engine 118 may determine that multiple conformations of the protein molecule are able to stably bind to the compound.

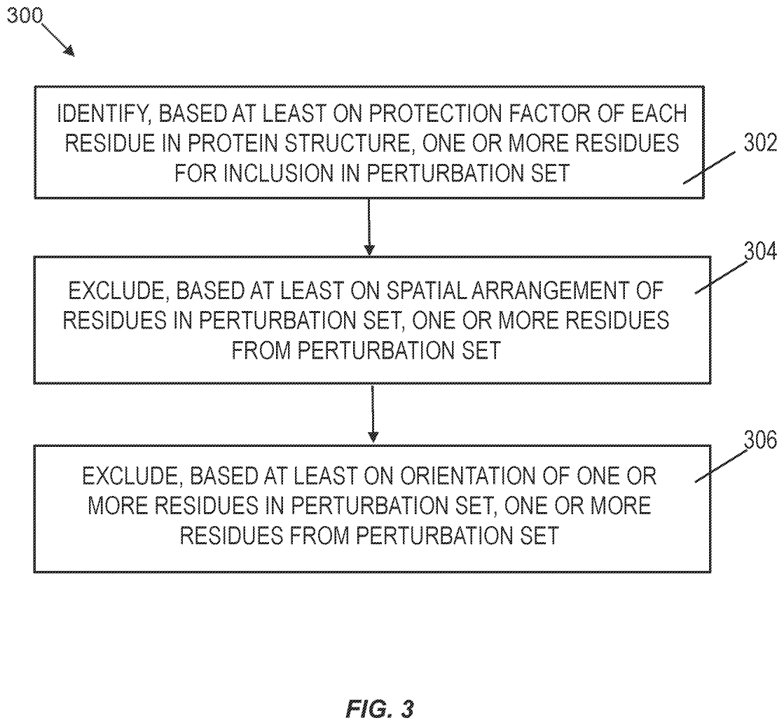

FIG. 3 depicts a flowchart illustrating an example of a process 300 for determining the perturbation set, in accordance with some example embodiments. Referring to FIGS. 1, 2A-B, and 3, the process 300 can be performed by the analysis controller 110, for example, the perturbation set engine 112, to determine a perturbation set for a particular conformation of a protein molecule. In some cases, the process 300 may implement operation 202 of the process 200 as well as operations 252 and 254 of the process 250.

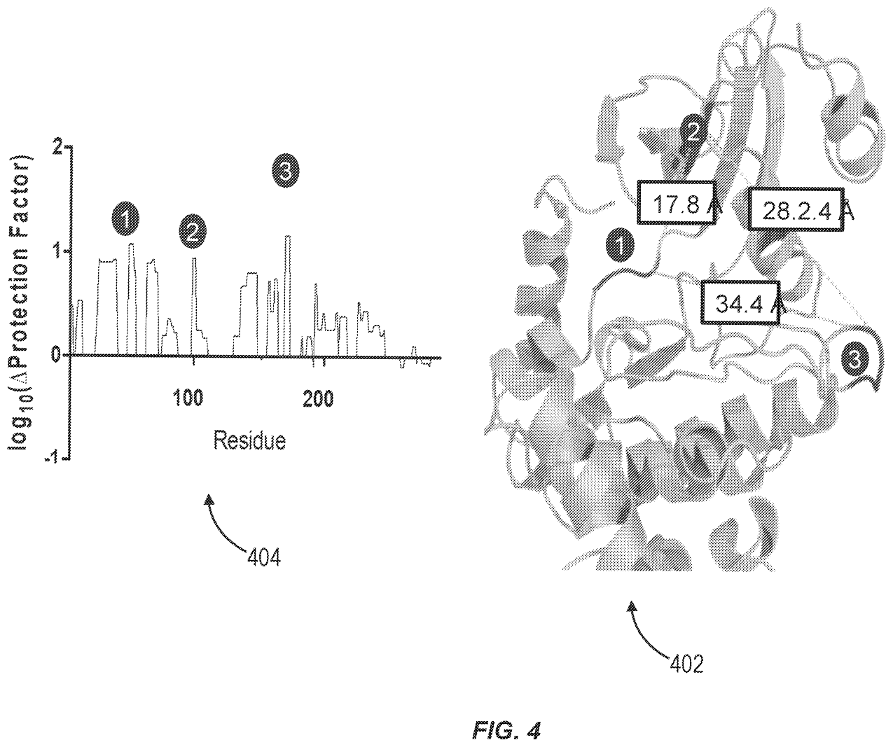

At 302, the analysis controller 110 may identify, based at least on a protection factor of each residue in a conformation of a protein molecule, one or more residues for inclusion in a perturbation set. In some example embodiments, the analysis controller 110, for example, the perturbation set engine 112, may generate a perturbation set, such as the first perturbation set 113a or the second perturbation set 113b, by at least identifying one or more residues of a plurality of regions in the corresponding protein conformation that exhibit a protection factor satisfying one or more thresholds. For example, in some cases, the perturbation set may be determined by including, in the perturbation set, one or more residues from the protein molecule exhibiting an above-threshold protection factor. FIG. 4 illustrates an example of a protein conformation 402 and a protection factor plot 404 that depicts the value of the protection factor associated with each residue in the protein conformation 402. The protection factor plot 404 includes values of the protection factors as a function of residues in the protein conformation 402. As noted, in some cases, one or more residues whose protection factor satisfies one or more thresholds can be included in the perturbation set. In some cases, the one or more thresholds may include a minimum protection factor value, a maximum protection factor value, and/or the like. Alternatively, the one or more thresholds may include a percentage of a maximum protection factor value of the residues in the protein molecule (e.g., 70% of the maximum protection factor value). In some cases, the perturbation set engine 112 may generate the perturbation set by including, in the perturbation set, a threshold quantity of residues (e.g., a maximum quantity of residues, a minimum quantity of residues, and/or the like). It should be appreciated that the quantity of residues included in the perturbation set, including the aforementioned threshold quantity of residues, may be fixed or variable between rounds of molecular dynamics simulation (MD). In the example shown in FIG. 4, three residues in the protein conformation 402 (labelled as 1, 2 and 3) are identified as being associated with an above-threshold protection factor and are thus identified by the perturbation set engine 112 for inclusion in the perturbation set.

At 304, the analysis controller 110 may exclude, based at least on a spatial arrangement of residues in the perturbation set, one or more residues from the perturbation set. In some example embodiments, the determining of the perturbation set can include excluding, from the perturbation set, one or more residues based on a spatial arrangement of the one or more residues in the protein conformation. For example, the analysis controller 110, for example, the perturbation set engine 112 may exclude a first residue if the distance between the first residue and a second residue in the protein conformation exceeds one or more dimensions of the compound. In some cases, for instance, the perturbation set engine 112 may exclude the first residue if the Euclidean distance between the nearest two atoms on first residue and the second residue exceeds the largest dimension of the compound.

In some cases, if the compound bound to the protein conformation at the residues included in the perturbation set dissociates, for example, after one or more rounds of molecular dynamics simulation (MD), the perturbation set engine 112 may rebuild the perturbation set to include the second residue but not the first residue. In some cases, the analysis controller 110 may further refine the perturbation set with the first residue (and/or the rebuilt perturbation set with the second residue) by at least excluding one or more residues whose orientation prevents them from forming a stable binding with the compound. Moreover, in some cases, one or more subsequent rounds of molecular dynamics simulations may be performed to assess the stability of the binding formed by the residues in each perturbation set. In doing so, the perturbation set engine 112 may generate, for each conformation of the protein molecule, a perturbation set that may be, in some cases, rebuilt based on the results of molecular dynamics simulation (MD).

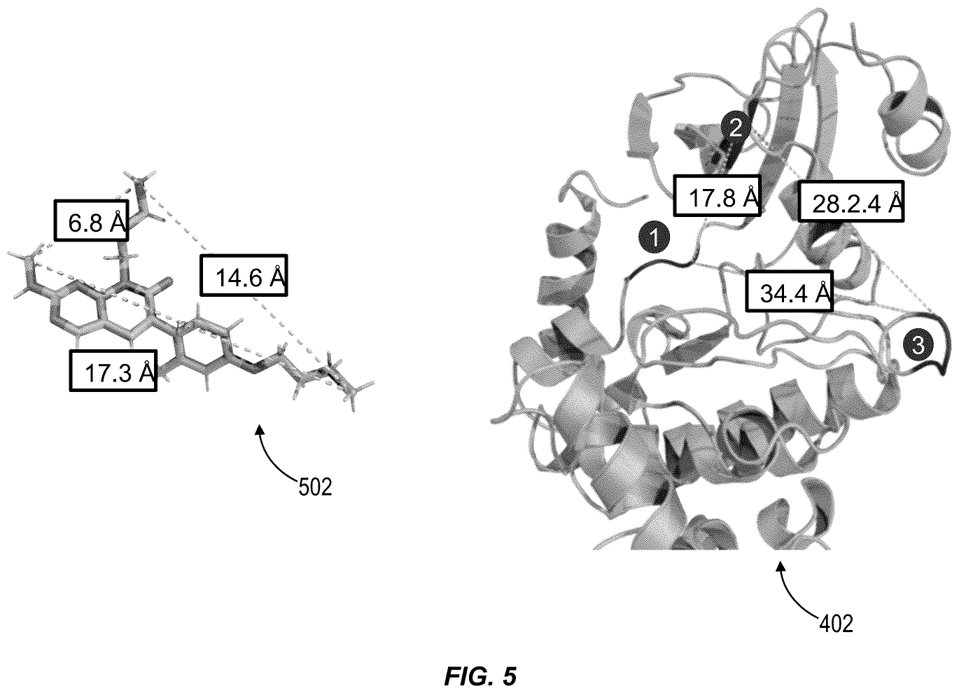

FIG. 5 depicts an example of a compound 502 and the protein molecule 402 including the three residues (labeled 1, 2 and 3) that was identified for inclusion in the perturbation (e.g., at operation 302). The compound 502 is characterized by lengths L1, L2 and L3 along three axis (e.g., orthogonal axis). If the lengths L1, L2 and L3 (or the largest of the lengths L1, L2, and L3) are smaller than the distance (e.g., the Euclidean distance) between, for example, residue cluster 1 and residue cluster 3, the compound 502 may be incapable of simultaneously binding to the residues in both residue cluster 1 and residue cluster 3. Likewise, if the lengths L1, L2, and L3 (or the largest of the lengths L1, L2, and L3) are smaller than the distance (e.g., the Euclidean distance) between residue clusters 2 and 3, the compound 502 may be incapable of simultaneously binding to both residues in residue cluster 2 and residue cluster 3). These observations may indicate that a single perturbation set cannot include residues in both residue cluster 1 and residue cluster 3 or residues in both residue cluster 2 and residue cluster 3. Contrastingly, where the distance (e.g., the Euclidean distance) between residue cluster 1 and residue cluster 2 is less than at least one of the lengths L1, L2 or L3, every residue in residue cluster 1 and residue cluster 2 may be included in the perturbation set.

In some cases, the analysis controller 110 will instruct the perturbation set engine 112 to identify clusters of residues for inclusion in and/or exclusion from the perturbation set in addition to, or instead of excluding residues on the basis of compound dimensions (with an additional constant in some cases, to allow for flexibility of the protein). For example, in some cases, residues in the protein molecule can be grouped into clusters, for example, by applying a clustering technique before one or more clusters of residues are excluded from the perturbation set. In addition to or instead of excluding individual residues from the perturbation set, the perturbation set engine 112 may identify clusters of residues for exclusion from the perturbation set in order to account for neighborhoods of adjacent residues that are capable and/or incapable of binding to the compound due to the physical dimensions of the compound. As described in more details below, a first cluster of residues may be identified for exclusion based on the quantity and location of residues included in cluster and the centroid of the cluster.

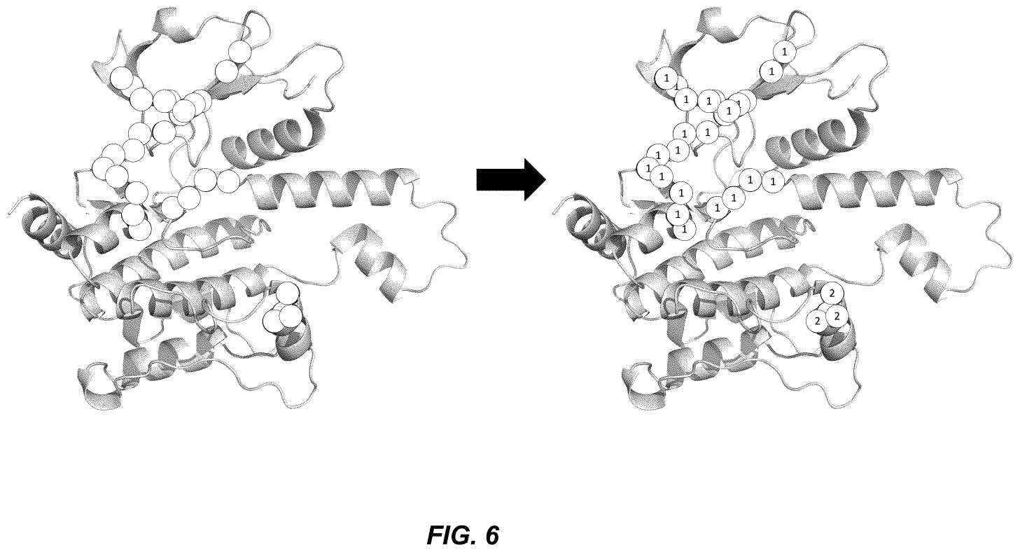

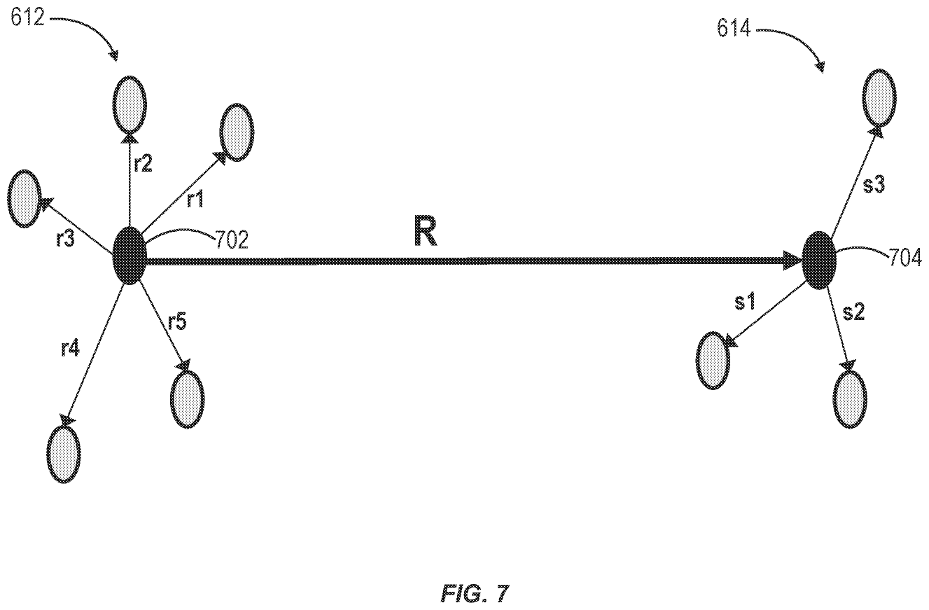

Examples of the clustering technique can include one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering. FIG. 6A depicts the residues in a protein conformation prior to clustering while FIG. 6B depicts the residues in the protein conformation subsequent to clustering. As illustrated in FIG. 6A, the residues in the protein conformation are represented by individual spheres. The results of applying the clustering technique, which are shown in FIG. 6(B), includes a first cluster 612 of residues denoted by spheres labeled with “1” and a second cluster 614 of residues denoted by spheres labeled with “2”. FIG. 7 depicts a schematic illustration of the first cluster 612 and the second cluster 614. The first cluster 612 has a first characteristic location 702 and the second cluster 614 has a second characteristic location 704. The first characteristic location 702 and the second characteristic location 704 can be a centroid of the first cluster 612 and the second cluster 614, respectively. The first cluster 612 can be characterized by a first mean distance d1, and the second cluster 614 can be characterized by a second mean distance d2 where

$$d 1 = \frac{{\sum }_{i}^{n} \vert {d}_{i} \vert}{n} and$$

$$d 2 = \frac{{\sum }_{i}^{m} \vert {s}_{i} \vert}{m}$$

wherein n denotes the quantity of residues in the first cluster 612, and m denotes the quantity of residues in the second cluster 614; |dᵢ| is the distance between the i-th residue in the first cluster 612 and the centroid 702 of the first cluster 612; and |sᵢ| is the distance between the i-th residue in the second cluster 614 and the centroid 704 of the first cluster 612. A displacement vector R indicates the displacement of the second centroid 704 relative from the first centroid 702. The distance between the two centroids can be described as the absolute value of the displacement vector R. Based on the foregoing the second cluster 614 can be excluded if the second cluster 614 has fewer residues than the first cluster 612, and if the absolute value of displacement vector R is greater than the sum of the first mean distance d1 and the second mean distance d2 (or |R|>(d1+d2)×constant).

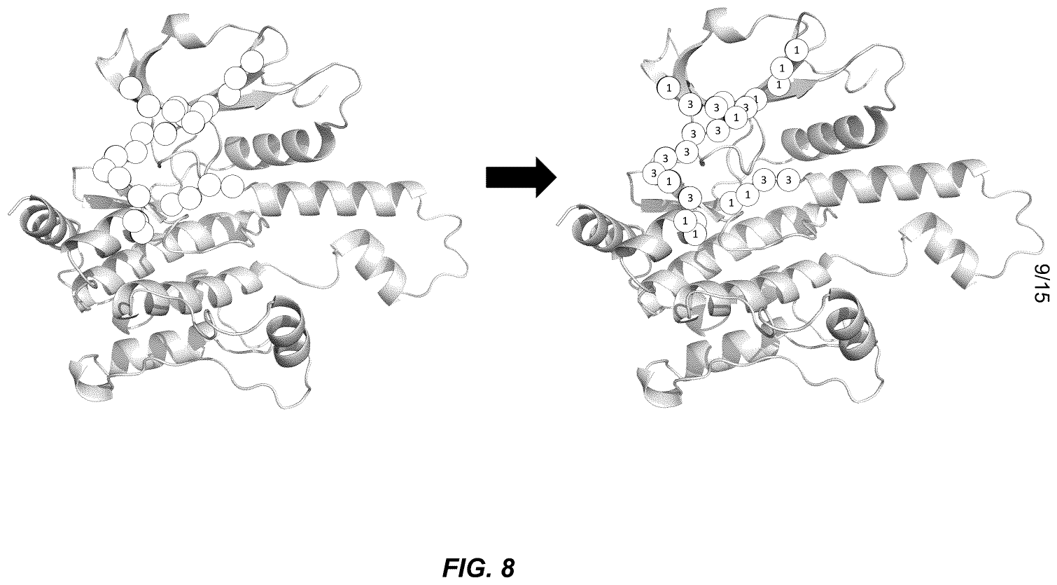

In addition to the exclusion of the second cluster 614, one or more individual residues within the first cluster 612 (e.g., selected for inclusion after the exclusion of the second cluster 614) can be further excluded from the perturbation set based on the orientation of one or more residues in the first cluster 612. For example, the residues in the first cluster 612 denoted by the spheres labeled “3” in FIG. 8 were kept in the perturbation set while those residues denoted by the spheres labeled “1” were selected for exclusion based on the orientation of these residues. In some cases, the residues denoted by the spheres labeled “1” may be excluded from the perturbation set based at least on those residues being required to undergo a threshold quantity of reorientations in order to bind with the compound. This threshold quantity of reorientations may be satisfied if the quantity of atoms in the residue required to undergo reorientation satisfies one or more thresholds. For instance, some of the residues denoted by a sphere labeled “1” may include a threshold quantity of atoms that are required to undergo reorientation in order to interact with the compound. Alternatively, and/or additionally, some of the residues denoted by a sphere labeled “1” may include one or more atoms whose angle of reorientation and/or distance of reorientation satisfy one or more thresholds. In some cases, some of the residues denoted by a sphere labeled “1” may include a sidechain that is required to undergo a threshold quantity of reorientations in order to bind with the compound. In these instances, the threshold quantity of reorientations may be satisfied if the angle between a first vector (defined by a first location of the center of geometry of the perturbation set containing the residue and a second location of the α-carbon (c_{α})) and a second vector (defined by the second location of the α-carbon (c_{α}) and a third location of the β-carbon (c_{β}), δ-carbon (c_{δ}), or γ-carbon (c_{γ}) in the residue) satisfies one or more thresholds.

Returning to FIG. 3, at 306, the analysis controller 110 may exclude, based at least on the orientation of one or more residues in the perturbation set, the one or more residues from the perturbation set. In some example embodiments, the determining of the perturbation set can further include excluding one or more residues (e.g., from the plurality of residues selected at operation 302, from the plurality of residues excluding those identified at operation 304, and/or the like) based at least on the orientation of the one or more residues and/or one or more constituent atoms in the one or more residues. For example, in some cases, a residue can be excluded from the perturbation set if the orientation of the residue requires a threshold quantity of changes in order to interact with the compound. An example computation for identifying a residue for exclusion based on its orientation and/or the orientation of its constituent atoms is shown in FIG. 10. In some cases, the aforementioned threshold quantity of changes may be satisfied if the quantity of atoms in the residue oriented away from the compound (e.g., oriented at an angle above a threshold angle relative to the compound) satisfy one or more thresholds (e.g., exceeds a certain number). The residue may be excluded in this case because a binding the compound and the second residue will require additional energy in order to reorient the residue. According, that residue may have a lower likelihood of forming a stable binding with the compound.

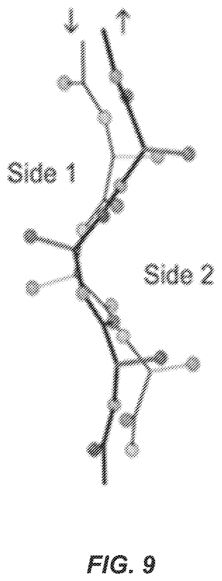

FIG. 9 illustrates an exemplary portion 900 of a protein conformation having multiple residues. As shown in FIG. 9, the sidechains of some residues in the protein conformation are oriented towards a first side (side 1) while the side chains of other residues in the protein conformation are oriented towards a second side (side 2). If the compound approaches the protein molecule from the first side (side 1), the likelihood of an interaction (or binding) between the compound and the residues whose sidechains are oriented towards the second side (side 2) of the protein molecule is small. Similarly, if the compound approaches the protein molecule from the second side (side 2), the likelihood of an interaction (or binding) between the compound and the residues whose sidechains are oriented towards the first side (side 1) is small.

FIG. 10 illustrates an exemplary method of excluding or including a residue based on the orientation of the residue. FIG. 10 illustrates an exemplary residue 1000 having a single α-carbon (denoted c_{α}) that is disposed at a certain location (e.g., with the spatial coordinate (x, y, z)). The residue 1000 may be a part of a perturbation set containing one or more other residues. Each residue in the perturbation set, including the residue 1000, may be associated with two directional unit-vectors (e.g., vectors having a magnitude of 1). For example, if the residue 1000 is considered the i-th residue in the perturbation set, the residue 1000 may be associated with a first unit vector (denoted as \vec{aₗ} in FIG. 10) that points from the center of geometry (COG) of the perturbation set towards the α-carbon (c_{α}) of the residue 1000 as well as a second unit vector (labeled \vec{bₗ} below, or as \vec{c_{α}c_{δ}} in FIG. 10) that points from the α-carbon (c_{α}) of the residue 1000 towards the δ-carbon (if the δ-carbon exists), the γ-carbon of the residue 1000 (if the γ-carbon exists), or the β-carbon of the residue 1000 (if the β-carbon exists). If the residue 1000 is a glycine, which does not contain α-carbon, the residue 1000 may be included in the perturbation set without being subject to any orientation-based exclusions. Otherwise, if the residue 1000 is not a glycine, the residue 1000 may be kept in the perturbation set if the following limitation is satisfied:

$$θ \leq arccos \vec{{a}_{ι}} \cdot \vec{{b}_{ι}} \leq 2 π - θ wherein , 0 \leq θ \leq π ( e . g . , θ = \frac{π}{4} ) .$$

In some cases, the residue 1000 may not have the second unit vector bᵢ (or \vec{c_{α}c_{δ}} in FIG. 10) (e.g., such as the case for glycine), in which case the residue 1000 may also be kept in the perturbation set without being subjected to orientation-based exclusions.

In some cases, the center of geometry (COG) for the perturbation set as a whole may be defined by the spatial coordinates (e.g., the three-dimensional coordinates (x, y, z)) of the backbone nitrogen atom (N) of each residue included in the perturbation set. It should be appreciated that the center of geometry (COG) for the perturbation set may be defined per frame of a molecular dynamics simulation (MD) trajectory t. Accordingly, the center of geometry [symbol] of a perturbation set for a frame in the trajectory t of a molecular dynamics simulation (MD) may be determined based on the equation below, in which [symbol] denotes a vector from the origin of the frame of reference to the i-th residue's backbone nitrogen atom, rᵢ in the perturbation set and N is the quantity of residues in the perturbation set.

$${\vec{C}}_{t} = \frac{\sum {\vec{r}}_{i , t}}{N}$$

FIG. 11 illustrates an exemplary schematic of the analysis controller 110, which includes the perturbation set engine 112 and the molecular dynamics simulation engine 114 as well as a first database 1104 and a second database 1108. The molecular dynamics simulation engine 114 can receive, from the first database 1104, at least a first structural information of the first conformation 119a of the protein molecule, and the first perturbation set 113a associated with the first conformation 119a generated by the perturbation set engine 112.

The first database 1104 (also referred to as unbound protein structure database) can include structural information (e.g., an unbound coordinate file) for multiple conformations (or conformers) of the protein molecule. The structural information for a protein conformation can indicate, for example, the shape of the protein conformation including by specifying the spatial arrangement of the constituent atoms. As described in more detail below, various conformations of the protein molecule can be generated by a protein structural information generating algorithm (e.g., an accelerated molecular dynamics algorithm and/or the like), which simulates the time evolution of the protein molecule (e.g., dynamics of the protein), and capture a structural configuration (e.g., the spatial arrangement of individual residues and/or the constituent atoms) of the protein molecule at various time instances during that particular trajectory. The structural configuration of each individual protein conformation and the corresponding perturbation set are provided as inputs to the molecular dynamics simulation (MD) engine 114.

The perturbation set engine 112 can receive the first structural information of the first conformation 119a (e.g., from the first database 1104) and generate the first perturbation set 113a. As noted, the perturbation set engine 112 may identify one or more residues in the first conformation 119a of the protein molecule for inclusion in the first perturbation set 113a based at least on the protection factor, spatial arrangement, and/or orientation of the residues in the first conformation 119a of the protein molecule (e.g., by performing the process 300 shown in FIG. 3). In instances where the perturbation set engine 112 generates the first perturbation set 113a by excluding one or more clusters of residues in the first conformation 119a of the protein molecule, the analysis controller 110 can further include a clustering engine that partitions the residues In the protein molecule into one or more clusters. The molecular dynamics simulation engine 114 can perform one or more molecular dynamic simulations based on the first structural information of the first conformation 119a of the protein molecule and the first perturbation set 113a while the assessment engine 118 can determine, based at least on the results of the molecular dynamics simulations, whether the residues in the first perturbation set 113a are capable of forming a stable binding between the compound and the protein molecule.

FIG. 12 illustrates an exemplary implementation of the analysis controller 110, in accordance with some example embodiments. As shown in FIG. 12, in some cases, the molecular dynamics simulation engine 114 can include a guided docking system that can receive the first perturbation set 113a (e.g., from the perturbation set engine 112) and structural information (e.g., an unbound coordinate file) for the first conformation 119a of the protein molecule in an unbound state (e.g., from the first database 1104). The guided docking system can generate a complex coordinate file that includes the structural information of the coupled protein-structure-compound, which in this case refers to the compound (e.g., ligand) bound to the first conformation 119a of the protein molecule. For example, in some cases, the complex coordinate file may be generated by clustering the individual frames of a molecular dynamics simulation (MD) based on the positions of the atoms in the residues included in the first perturbation set 113a and selecting one or more clusters based on the quantity of atoms contained therein (e.g., most populated clusters and/or the like).

It should be appreciated that other conformations of the protein molecule in the first database 1104 (e.g., the unbound protein structure database), such as the second conformation 119a of the same protein molecule, can be provided as an input to the guided docking system before undergoing guided docking to generate one or more corresponding complex coordinate files. As a single protein molecule is capable of assuming multiple conformations, a single protein molecule may be associated with multiple unbound coordinate files and complex coordinate files (e.g., one complex coordinate file for each unbound coordinate file).

In some cases, the unbound coordinate file can specify the shape of a particular conformation of the protein molecule, such as the first conformation 119a, by least specifying the position and orientation of every atom in the protein molecule. The guided docking system can generate the complex coordinate file by reducing (e.g., minimizing) an energy function associated with the compound bound to the first conformation 119a of the protein molecule while the distance from the residues in the first perturbation set 113a can be used in the scoring function (e.g., Rosetta Dock, HADDOCK, Autodock, and/or the like). The resulting complex coordinate file may specify the locations of each atom in the coupled protein-structure-compound including, for example, the atoms in the first conformation 119a of the protein molecule and the atoms in the compound.

The molecular dynamics simulation engine 114 can include a temperature searching system that can receive the complex coordinate file (e.g., including structural information of the coupled compound-protein-structure) from the guided docking system and simulate the temporal evolution of the corresponding coupled protein-structure-compound. The temperature searching system can simultaneously receive multiple complex coordinate files where each complex coordinate file includes structural information of compound bound to a different conformation of the protein molecule. In some cases, the temperature searching system may perform multiple arounds of molecular dynamic simulation (MD), for example, at different temperatures and for different coupled compound-protein-structures (e.g., complexes formed by the compound bound to different conformations of the same protein molecule) in order to determine a temperature at which a threshold quantity of coupled compound-protein-structures dissociate.

The temporal evolution of multiple coupled protein-structure-compounds (e.g., generated for different conformations of the protein molecule) can be simulated at a first temperature (e.g., 375 Kelvin). This can result in multiple simulations (or trajectories) of each coupled protein-structure-compound. For example, three simulations or trajectories can be simulated for each coupled compound-protein-structure at the first temperature, with each simulation (or trajectory) being performed for a first predetermined time period (e.g., 100 nanoseconds). If none of the trajectories results in the disassociation of the coupled compound-protein-structure, one or more additional rounds of molecular dynamic simulations may be performed, with each simulation (or trajectory) being performed for a second predetermined time period that is longer than the first predetermined time period (e.g. 1 microsecond) and/or at a second temperature that is higher than the first temperature (e.g., 25 Kelvin higher than the first temperature).

Alternatively, if every molecular dynamics simulation (MD) or (trajectory) calculated at a given temperature results in the dissociation of the coupled compound-protein-structure, one or more additional rounds of molecular dynamics simulation can be performed at a second temperature lower than the first temperature (e.g., 25 Kelvin lower than the first temperature) and typically for 100 nanoseconds.

Referring again to FIG. 12, a boundary test system can determine whether a coupled protein-structure-compound has dissociated, for example, during or at the end of a round of molecular dynamics simulation (or trajectory). The boundary test system can calculate a distance metric indicative of a distance between a first center of geometry (COG) of the first perturbation set 113a and a second center of geometry (COG) of the compound during the course of the molecular dynamics simulation. In some cases, the distance metric may correspond to the distance between a center of geometry (COG) of the backbone nitrogen (N) atoms in the first perturbation set 113a and the center of geometry (COG) of the compound. If the value of the distance metric satisfies one or more thresholds (e.g., exceeds a threshold distance value), the coupled protein-structure-compound is considered to be dissociated.

In some cases, a complex coordinate file may be generated for each coupled compound-protein-structure in which the protein molecule assumes a different conformation. Each complex coordinate file can then undergo multiple rounds of molecular dynamics simulation (or trajectories) at the temperatures determined acceptable by the temperature searching system. In some cases, a new complex coordinate file can include structural information associated with the coupled protein-structure-compound after (or during) the time evolution simulated by the temperature searching system at the first temperature. If the corresponding coupled compound-protein-structure did not dissociate at the end of the molecular dynamics simulation, the corresponding complex coordinate file can be stored in a bound structure database, and can be further evaluated by the assessment engine 118 including, for example, by calculating one or more metrics as a proxy for agreement with data between the compound and the protein molecule (e.g., seeking to maximize satisfaction score and minimize the divergence score, and/or the like). Alternately, the complex coordinate file associated with a coupled compound-protein-structure that dissociates during and/or at the conclusion of one or more rounds of molecular dynamics simulation can be discarded. Moreover, in some cases, the unbound coordinate file specifying the conformation of a protein molecule conformation when the protein molecule dissociated from the compound may be returned to the unbound protein structure database (e.g., the first database 1104) such that the dissociated conformation of the protein molecule may undergo additional evaluation.

A satisfaction score and a divergence score can be calculated by the assessment engine 118 and assigned to the complex coordination file of the corresponding coupled compound-protein-structure. The satisfaction score can be indicative of the strength of the bond between the compound and a particular conformation of the protein molecule in a corresponding compound-protein-structure. In some cases, the satisfaction score can be a function of (or proportional to) the number of hydrogen bonds and/or the packing density of atoms in the coupled compound-protein-structure at or around one or more residues in the protein molecule. A higher value of the satisfaction score may be desirable as it can indicate conformity between the molecular dynamic simulation results and experimental results.

The satisfaction score for a coupled compound-protein-structure can be calculated by the following expression if the coupled compound-protein-structure did not dissociate during or at the conclusion of the molecular dynamics simulations:

$$Satisfaction Score = ( 〈 \overline{{N}_{c}} 〉 〈 \overline{{N}_{H}} 〉 - Δ E ) / 〈 \overline{d} 〉$$

Alternatively, the satisfaction score for the coupled compound-protein-structure can be determined based on the following equation if the coupled compound-protein-structure did dissociate during or at the conclusion of the molecular dynamics simulations:

$$Satisfaction Score = ( 〈 \overline{{N}_{c}} 〉 〈 \overline{{N}_{H}} 〉 - Δ E ) / {d}_{max}$$

In some example embodiments, ΔE is an energy value, determined for the trajectory of the protein molecule with and without the compound bound; N_{C} is the number of heavy atoms within 6.5 A of an amide nitrogen atom of a residue in the perturbation set for any particular frame (or time instance) of a molecular dynamics simulation; N_{H} is the number of nitrogen and oxygen atoms within 2.5 A of an amide nitrogen atom of the residue in the perturbation set for any particular frame of the molecular dynamics simulation; d is the distance from an amide nitrogen of any particular residue in the perturbation set to the center of geometry (COG) of the compound (e.g., ligand) for any particular frame of the molecular dynamics simulation; [symbol] variable [symbol] is the mean of that v̅a̅r̅i̅a̅b̅l̅e̅ of the molecular dynamics simulation (e.g., over the various frames of the MD simulation).

A divergence score can be calculated by the following expression:

Divergence Score=

[symbol]

where [symbol] is the standard deviation in the mean distance from amide nitrogen atoms (e.g., nitrogen (N) atoms contained in amide groups) in the perturbation set to the center of geometry (COG) of the compound (e.g., ligand) over a trajectory. More generally, during the course of the molecular dynamics simulation (e.g., from one frame in the corresponding trajectory to another), the distance between the amide nitrogen of a residue and the center of mass of the compound may vary. A mean distance value can be calculated from the various distance values (associated with different frames). Based on the mean value and the various distance value, a standard deviation value (or the divergence score) can be determined. The divergence score can be high during earlier rounds of molecular dynamics simulation but will often decrease over subsequent rounds in instances where the compound is properly bound in a well-formed protein conformation.

In some cases, the mean distance (or divergence) between the compound and the protein molecule may be determined based on the center of geometry (COG) of the atoms in the compound and the center of geometry (COG) of residues in the perturbation set, or on the mean distance (or divergence) between the center of geometry of the compound and the each of the backbone nitrogen atoms in perturbation set residues (as is described below). The center of geometry (COG) of the residues in the perturbation set may be determined based on the locations of the backbone nitrogen (N) atoms in those residues. It should be appreciated that each residue in the perturbation set will have one backbone nitrogen (N) atom. To further illustrate, take [symbol] to denote the center of geometry of the collection of atoms forming the compound, and [symbol] to denote the spatial coordinates (e.g., (x, y, z)) of each backbone nitrogen (N) atom in the perturbation set. The mean distance, d̅ₜ, may correspond to the average of the individual distances, d_{i,t}, in dₜ for any frame of the simulation t. The divergence score [symbol] may be determined based on the equations below.

$${d}_{t} = {\vert \vec{C} - {\vec{n}}_{i} \vert}_{t}$$

$${\overline{d}}_{t} = {N}^{- 1} \sum {d}_{i , t}$$

$$\overline{{σ}_{\overline{〈 d 〉}}} = \sqrt{{M}^{- 1} \sum {( 〈 \overline{d} 〉 - {\overline{d}}_{t} )}^{2}}$$

wherein N denotes the number of residues in the perturbation set, M denotes the number of frames in a trajectory, and |v̅a̅r̅i̅a̅b̅l̅e̅|=\sqrt{x²+y²+z²}, or the magnitude of the variable in the units used above.

The molecular dynamics simulation engine 114 can include a determination system to determine whether to perform additional rounds of molecular dynamics simulation (MD), for example, at different conditions. For example, if a threshold quantity of trajectories for different starting poses of a particular conformation of the protein molecule end in the corresponding coupled compound-protein-structure disassociating at a given temperature, one or more additional rounds of molecular dynamics simulation from the same conformation can be performed at lower temperatures (e.g., 25K lower than the initial simulations). Similarly, if a threshold quantity of trajectories for a threshold portion of different starting poses of the protein conformation remains associated for a particular round, the temperature may be elevated to favor dissociation in one or mor subsequent rounds of molecular dynamics simulation.

In some cases, multiple trajectories may not result in the dissociation of the coupled compound-protein-structure while the corresponding satisfaction score is less than a threshold (e.g., less than 50, the actual score will always be relative to the size of the perturbation set, among other things, and this threshold will be case dependent) and/or the corresponding divergence score is greater than a threshold (e.g., less than 1, this is absolute and will not typically be case-dependent). In this case, one or more of the corresponding complex coordinate files can be stripped to generate additional conformations of the protein molecule for storage in the unbound protein structure database (e.g., the first database 1104). These conformations can be different from the conformations of the protein molecule provided to the molecular dynamics simulation engine 114 to perform one or more other rounds (e.g., one or more previous rounds) of molecular dynamics simulation after a new perturbation set has been generated and provided to the docking algorithm. Structural information associated with these conformations of the protein molecule can be provided as input to the guided docking system of the molecular dynamics simulation engine 114. For example, one or more rounds of molecular dynamic simulations can be performed by the molecular dynamics simulation engine 114 for each additional conformation of the protein molecule and the corresponding perturbation set to determine if the perturbation set is capable of forming a stable binding between the compound and the protein molecule across different conformations of the protein molecule. For example, a particular conformation of the protein molecule (e.g., the corresponding structural information such as the unbound coordinates file) and the corresponding perturbation set can be provided to the guided docking system to generate a complex coordinate file for the compound bound to that conformation of the protein molecule at the residues included in the perturbation set. If the molecular dynamics simulation engine 114 is provided with a complex coordinate file (e.g., of that protein conformation coupled with the compound), the guided docking system can be bypassed, and the complex coordinate file can be provided to the temperature searching system.

The determination system can determine that the plurality of regions in the first perturbation set 113a (used by the molecular dynamics simulation engine 114 to perform the simulations) is identified as forming a stable binding between the compound and the protein molecule. For example, the determination system can determine that a complex coordinate file should be placed in the bound structure database and no additional simulations are needed for that molecule when one or more exit criteria are met. In some cases, the exit criterion can be based on maximal satisfaction scores and minimal divergence scores of one or more trajectories. For instance, if a trajectory satisfies a satisfaction score criterion (e.g., the satisfaction score has plateaued or stopped increasing) and a divergence score criterion (e.g., the divergence score is less than 1), then this complex coordinate file will be placed into the bound structure database.

In some cases, the exit criterion can also be based on alignment between a threshold quantity of structures (e.g., at least three or a different threshold) in the bound structure database. For example, the atoms of the protein molecule in three (or more) complex coordinate files (e.g., associated with a first trajectory and a second trajectory, respectively) can be aligned, and the locations of the atoms of the ligand in the first new complex coordinate file can be compared with the atoms of the ligand in the second and third new complex coordinate file. This may be achieved, for example, by first taking the intersection of sets formed by selecting all atoms within a predefined distance to the ligand (e.g., 6 angstrom). The atoms in the two complex coordinate files may be aligned using carbon alpha root mean square distance (RMSD) minimization of the intersection of sets. From these coordinates, one can calculate an all-atom root mean square distance (RMSD) between the atoms in the ligand between each complex coordinate file. If that value is lower than some predefined threshold (e.g., 1 angstrom²) for the threshold quantity (e.g., three or more) complex coordinate files located in the bound structure database then the exit criteria has been satisfied and those coupled compound-protein-structures are placed in the result database.

In some example embodiments, once the exit criteria are satisfied, the residues in the perturbation set used in the current simulation (e.g., the first perturbation set 113a) is identified as forming a stable binding between the compound and the protein molecule. Additionally, the perturbation set and the structural information of the protein conformation used in the current simulation (e.g., the first perturbation set 113a, the first structural information, etc.) are stored in the second database 1108 (also referred to as the selected perturbation set database).

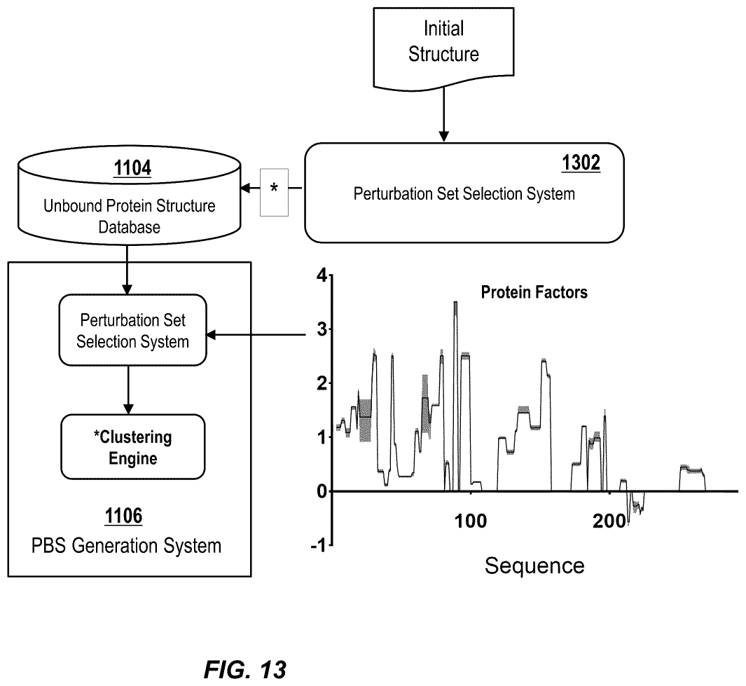

FIG. 13 illustrates an exemplary implementation of a protein structural information generating algorithm 1302 that can generate multiple conformations of the protein molecule and store the corresponding structural information files (e.g., unbound structural information) in the first database 1104 (e.g., the unbound protein structure database). It should be appreciated that unbound protein conformations, and the corresponding unbound structural information, can be generated once (e.g., at the beginning of the molecular dynamics simulation) and stored in the first database 1104 (e.g., the unbound protein structure database). In some cases, the first database 1104 (e.g., the unbound protein structure database) can store, for a single protein molecule, multiple conformations of the protein molecule that correspond to the structural evolution of the protein molecule over time. As described above, multiple conformations of the same protein molecule structure can be retrieved from the first database 1104 and provided as an input (e.g., simultaneously) to the guided docking system. The protein spatial structure generating algorithm 1302 can include an accelerated molecular dynamic simulator (often referred to as accelerated molecular dynamics simulation (MD), or metadynamics, etc.) that can receive the unbound structural information of the protein conformations and simulate time evolution of the initial three-dimensional structure.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

Item 2: The method of Item 1, wherein the protection factor associated with each residue in the first conformation corresponds to a difference between a first energy of a residue in a bound state and a second energy of the residue in an unbound state.

Item 3: The method of any of Items 1 to 2, further comprising: identifying, based at least on the protection factor of the one or more residues satisfying one or more thresholds, the one or more residues for inclusion in the first perturbation set.

Item 4: The method of Item 3, further comprising: determining that the protection factor of the one or more residues satisfies the one or more thresholds based at least on a value of the protection factor being within a percentile of a maximum protection factor value observed for the first conformation of the protein molecule.

Item 5: The method of Item 4, further comprising: determining the percentile based at least on a magnitude of a difference between the maximum protection factor value and a plurality of other protection factor values observed for the first conformation of the protein molecule.

Item 6: The method of any of Items 4 to 5, wherein the percentile is a value between 5 and 20.

Item 7: The method of any of Items 1 to 6, wherein the interaction between the compound and the first plurality of residues in the first perturbation set includes at least a portion of the first plurality of residues in the first perturbation set forming an induced fit pocket within the first conformation of the protein molecule.

Item 8: The method of any of Item 1 to 7, wherein each round of molecular dynamics simulation simulates a temporal evolution of a coupled protein-structure-compound including the compound bound to the first conformation of the protein molecule at the first plurality of residues included in the first perturbation set.

Item 9: The method of Item 8, wherein the coupled protein-structure-compound is generated based at least on the first perturbation set and a structural information specifying a spatial arrangement of atoms in the first conformation of the protein molecule in an unbound state.

Item 10: The method of any of Items 1 to 9, wherein the determining the first perturbation set further comprises excluding, from the first perturbation set, at least a third residue that requires a threshold quantity of reorientations in order to interact with the compound.

Item 11: The method of Item 10, wherein the threshold quantity of reorientations is satisfied by (i) a quantity of atoms in the third residue that require a reorientation in order to interact with the compound, (ii) an angle of reorientation that one or more atoms in the third residue is required to undergo in order to interact with the compound, and/or (iii) a distance of reorientation that the one or more atoms in the third residue is required to undergo in order to interact with the compound.

Item 12: The method of any of Items 10 to 11, further comprising: determining a first vector from a center of geometry (COG) of the first plurality of residues in the first perturbation set and an α-carbon of the third residue; determining a second vector from the α-carbon to one of a δ-carbon, γ-carbon, or β-carbon present in the third residue; and determining that the threshold quantity of reorientations is satisfied based at least on an angle formed by the first vector and the second vector satisfying one or more thresholds.

Item 13: The method of any of Items 10 to 12, wherein the third residue is kept in the first perturbation set based at least on the third residue being glycine.

Item 14: The method of any of Items 1 to 13, wherein the determining the first perturbation set further comprises determining a distance between a first centroid of a first cluster of residues and a second centroid of a second cluster of residues, determining that the distance fails to correspond to one or more dimensions of the compound, and excluding, from the first perturbation set, the first residue based at least on the first residue being a part of the first cluster of residues.

Item 15: The method of Item 14, wherein the determining the first perturbation set further comprises applying, to the first plurality of residues, a clustering technique to partition the first plurality of residues into at least the first cluster of residues and the second cluster of residues.

Item 16: The method of Item 15, wherein the clustering technique includes one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

Item 17: The method of any of Items 14 to 15, wherein the determining the first perturbation set further comprises determining a first quantity of residues in the first cluster of residues and a second quantity of residues in the second cluster of residues, determining a first mean distance between the first centroid and residues in the first cluster, and determining a second mean distance between the second centroid and residues in the second cluster.

Item 18: The method of Item 17, wherein the determining the first perturbation set further comprises excluding, from the first perturbation set, the first cluster of residues based at least on a determination that (i) the second cluster includes fewer residues than the first cluster, and (ii) the distance between the first centroid of the first cluster and the second centroid of the second cluster exceeds a sum of the first mean distance between the first centroid and residues in the first cluster and the second mean distance between the second centroid and residues in the second cluster.

Item 19: The method of any of Items 1 to 18, wherein the result comprises a distance between a first center of geometry of the compound and a second center of geometry of a plurality of backbone nitrogen (N) atoms in the first plurality of residues in the first perturbation set.

Item 20: The method of any of Items 1 to 19, wherein the result comprises a distance between a first center of geometry of the compound and one or more amide nitrogen atoms present in the first plurality of residues in the first perturbation set.

Item 21: The method of any of Items 1 to 20, wherein the one or more rounds of molecular dynamics simulation comprise a first round of molecular dynamics simulation performed at a first temperature and a second round of molecular dynamics simulation performed at a second temperature.

Item 22: The method of any of Items 1 to 21, wherein the one or more rounds of molecular dynamics simulation includes a first round of molecular dynamics simulation performed for a first length of time and a second round of molecular dynamics simulation performed for a second length of time.

Item 23: The method of any of Items 1 to 22, wherein the one or more rounds of molecular dynamics simulation includes a first round of molecular dynamics simulation performed for the first conformation of the protein molecule that is associated with the first perturbation set and a second round of molecular dynamics simulation performed for a second conformation of the protein molecule that is associated with a second perturbation set.

Item 24: The method of Item 23, wherein each of the first conformation and the second conformation comprises a three-dimensional structure having a different spatial arrangement of atoms in the protein molecule.

Item 25: The method of any of Items 23 to 24, further comprising: determining the second perturbation set for the second conformation of the protein molecule, the determining comprising at least one of (i) identifying, based at least on the protection factor of at least a portion of residues in the protein molecule, one or more residues for inclusion in the second perturbation set, (ii) excluding, from the second perturbation set, at least a third residue whose distance to a fourth residue in the second perturbation set fails to correspond to the one or more dimensions of the compound, and (iii) excluding, from the second perturbation set, at least a fifth residue that requires a threshold quantity of reorientations in order to interact with the compound.

Item 26: The method of any of Items 1 to 25, wherein the one or more rounds of molecular dynamics simulation includes a plurality of rounds of molecular dynamics simulation, and wherein each round of molecular dynamics simulation subjects a coupled compound-protein-structure including the compound bound to the protein molecule at the first plurality of residues in the first perturbation set to a different condition.

Item 27: The method of Item 26, wherein the result of the one or more rounds of molecular dynamics simulation includes one or more conditions in which the coupled compound-protein-structure disassociates.

Item 28: The method of Item 27, wherein the one or more conditions include a temperature at which the coupled compound-protein-structure dissociates.

Item 29: The method of any of Items 27 to 28, wherein the one or more conditions include a length of time after which the coupled compound protein-structure dissociates.

Item 30: The method of any of Items 1 to 29, wherein the one or more rounds of molecular dynamics simulation includes a round of molecular dynamics simulation that is performed at a higher temperature than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

Item 31: The method of any of Items 1 to 30, wherein the one or more rounds of molecular dynamics simulation includes a round of molecular dynamics simulation that is performed at a lower temperature than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

Item 32: The method of any of Items 1 to 31, wherein the one or more rounds of molecular dynamics simulation includes a round of molecular dynamics simulation that is performed for a longer time period than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

Item 33: The method of any of Items 1 to 32, wherein the one or more rounds of molecular dynamics simulation includes a round of molecular dynamics simulation that is performed for a shorter time period than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

Item 34: The method of any of Items 1 to 33, wherein the first plurality of residues in the perturbation set is identified as forming a stable binding by at least determining, based at least on the result of the one or more rounds of molecular dynamics simulations, one or more metrics quantifying a binding affinity between the compound and the first conformation of the protein molecule, and determining, based at least on the one or more metrics, the first plurality of residues in the perturbation set as forming the stable binding between the compound and the first conformation of the protein molecule.

Item 35: The method of any of Items 1 to 34, wherein the determining the first perturbation set further comprises determining a distance between the first residue and the second residue, and in response to determining that the distance between the first residue and the second residue exceeds the one or more dimensions of the compound, excluding the first residue from the first perturbation set.

Item 36: A system, comprising: at least one data processor; and at least one memory storing instructions, which when executed by the at least one data processor, result in operations comprising: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

Item 37: The system of Item 36, wherein the protection factor associated with each residue in the first conformation corresponds to a difference between a first energy of a residue in a bound state and a second energy of the residue in an unbound state.

Item 38: The system of any of Items 36 to 37, wherein the operation further comprise: identifying, based at least on the protection factor of the one or more residues satisfying one or more thresholds, the one or more residues for inclusion in the first perturbation set.

Item 39: The system of Item 38, wherein the operations further comprise: determining that the protection factor of the one or more residues satisfies the one or more thresholds based at least on a value of the protection factor being within a percentile of a maximum protection factor value observed for the first conformation of the protein molecule.

Item 40: The system of Item 39, wherein the operations further comprise: determining the percentile based at least on a magnitude of a difference between the maximum protection factor value and a plurality of other protection factor values observed for the first conformation of the protein molecule.

Item 41: The system of any of Items 39 to 40, wherein the percentile is a value between 5 and 20.

Item 42: The system of any of Items 36 to 41, wherein the interaction between the compound and the first plurality of residues in the first perturbation set includes at least a portion of the first plurality of residues in the first perturbation set forming an induced fit pocket within the first conformation of the protein molecule.

Item 43: The system of any of Items 36 to 42, wherein each round of molecular dynamics simulation simulates a temporal evolution of a coupled protein-structure-compound including the compound bound to the first conformation of the protein molecule at the first plurality of residues included in the first perturbation set.

Item 44: The system of Item 43, wherein the coupled protein-structure-compound is generated based at least on the first perturbation set and a structural information specifying a spatial arrangement of atoms in the first conformation of the protein molecule in an unbound state.

Item 45: The system of any of Items 36 to 44, wherein the determining the first perturbation set further comprises excluding, from the first perturbation set, at least a third residue that requires a threshold quantity of reorientations in order to interact with the compound.

Item 46: The system of Item 45, wherein the threshold quantity of reorientations is satisfied by (i) a quantity of atoms in the third residue that require a reorientation in order to interact with the compound, (ii) an angle of reorientation that one or more atoms in the third residue is required to undergo in order to interact with the compound, and/or (iii) a distance of reorientation that the one or more atoms in the third residue is required to undergo in order to interact with the compound.

Item 47: The system of any of Items 45 to 46, further comprising: determining a first vector from a center of geometry (COG) of the first plurality of residues in the first perturbation set and an α-carbon of the third residue; determining a second vector from the α-carbon to one of a δ-carbon, γ-carbon, or β-carbon present in the third residue; and determining that the threshold quantity of reorientations is satisfied based at least on an angle formed by the first vector and the second vector satisfying one or more thresholds.

Item 48: The system of any of Items 45 to 47, wherein the third residue is kept in the first perturbation set based at least on the third residue being glycine.

Item 49: The system of any of Items 36 to 48, wherein the determining the first perturbation set further comprises determining a distance between a first centroid of a first cluster of residues and a second centroid of a second cluster of residues, determining that the distance fails to correspond to one or more dimensions of the compound, and excluding, from the first perturbation set, the first residue based at least on the first residue being a part of the first cluster of residues.

Item 50: The system of Item 49, wherein the determining the first perturbation set further comprises applying, to the first plurality of residues, a clustering technique to partition the first plurality of residues into at least the first cluster of residues and the second cluster of residues.

Item 51: The system of Item 50, wherein the clustering technique includes one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

Item 52: The system of any of Items 49 to 51, wherein the determining the first perturbation set further comprises determining a first quantity of residues in the first cluster of residues and a second quantity of residues in the second cluster of residues, determining a first mean distance between the first centroid and residues in the first cluster, and determining a second mean distance between the second centroid and residues in the second cluster.

Item 53: The system of Item 52, wherein the determining the first perturbation set further comprises excluding, from the first perturbation set, the first cluster of residues based at least on a determination that (i) the second cluster includes fewer residues than the first cluster, and (ii) the distance between the first centroid of the first cluster and the second centroid of the second cluster exceeds a sum of the first mean distance between the first centroid and residues in the first cluster and the second mean distance between the second centroid and residues in the second cluster.

Item 54: The system of any of Items 36 to 53, wherein the result comprises a distance between a first center of geometry of the compound and a second center of geometry of a plurality of backbone nitrogen (N) atoms in the first plurality of residues in the first perturbation set.

Item 55: The system of any of Items 36 to 54, wherein the result comprises a distance between a first center of geometry of the compound and one or more amide nitrogen atoms present in the first plurality of residues in the first perturbation set.

Item 56: The system of any of Items 36 to 55, wherein the one or more rounds of molecular dynamics simulation comprise a first round of molecular dynamics simulation performed at a first temperature and a second round of molecular dynamics simulation performed at a second temperature.

Item 57: The system of any of Items 36 to 56, wherein the one or more rounds of molecular dynamics simulation includes a first round of molecular dynamics simulation performed for a first length of time and a second round of molecular dynamics simulation performed for a second length of time.

Item 58: The system of any of Items 36 to 57, wherein the one or more rounds of molecular dynamics simulation includes a first round of molecular dynamics simulation performed for the first conformation of the protein molecule that is associated with the first perturbation set and a second round of molecular dynamics simulation performed for a second conformation of the protein molecule that is associated with a second perturbation set.

Item 59: The system of Item 58, wherein each of the first conformation and the second conformation comprises a three-dimensional structure having a different spatial arrangement of atoms in the protein molecule.

Item 60: The system of any of Items 58 to 59, wherein the operations further comprise: determining the second perturbation set for the second conformation of the protein molecule, the determining comprising at least one of (i) identifying, based at least on the protection factor of at least a portion of residues in the protein molecule, one or more residues for inclusion in the second perturbation set, (ii) excluding, from the second perturbation set, at least a third residue whose distance to a fourth residue in the second perturbation set fails to correspond to the one or more dimensions of the compound, and (iii) excluding, from the second perturbation set, at least a fifth residue that requires a threshold quantity of reorientations in order to interact with the compound.

Item 61: The system of any of Items 36 to 60, wherein the one or more rounds of molecular dynamics simulation includes a plurality of rounds of molecular dynamics simulation, and wherein each round of molecular dynamics simulation subjects a coupled compound-protein-structure including the compound bound to the protein molecule at the first plurality of residues in the first perturbation set to a different condition.

Item 62: The system of Item 61, wherein the result of the one or more rounds of molecular dynamics simulation includes one or more conditions in which the coupled compound-protein-structure disassociates.

Item 63: The system of Item 62, wherein the one or more conditions include a temperature at which the coupled compound-protein-structure dissociates.

Item 64: The system of any of Items 62 to 63, wherein the one or more conditions include a length of time after which the coupled compound protein-structure dissociates.

Item 65: The system of any of Items 36 to 64, wherein the one or more rounds of molecular dynamics simulation includes a round of molecular dynamics simulation that is performed at a higher temperature than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

Item 66: The system of any of Items 36 to 65, wherein the one or more rounds of molecular dynamics simulation includes a round of molecular dynamics simulation that is performed at a lower temperature than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

Item 67: The system of any of Items 36 to 66, wherein the one or more rounds of molecular dynamics simulation includes a round of molecular dynamics simulation that is performed for a longer time period than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

Item 68: The system of any of Items 36 to 67, wherein the one or more rounds of molecular dynamics simulation includes a round of molecular dynamics simulation that is performed for a shorter time period than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

Item 69: The system of any of Items 36 to 68, wherein the first plurality of residues in the perturbation set is identified as forming a stable binding by at least determining, based at least on the result of the one or more rounds of molecular dynamics simulations, one or more metrics quantifying a binding affinity between the compound and the first conformation of the protein molecule, and determining, based at least on the one or more metrics, the first plurality of residues in the perturbation set as forming the stable binding between the compound and the first conformation of the protein molecule.

Item 70: The system of any of Items 36 to 69, wherein the determining the first perturbation set further comprises determining a distance between the first residue and the second residue, and in response to determining that the distance between the first residue and the second residue exceeds the one or more dimensions of the compound, excluding the first residue from the first perturbation set.

Item 71: A non-transitory computer readable medium storing instructions, which when executed by at least one data processor, result in operations comprising: (a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound; (b) performing one or more rounds of molecular dynamics simulations to generate a result indicative of an interaction between the compound and a first plurality of residues in the first perturbation set; and (c) identifying, based at least on the result of the one or more rounds of molecular dynamics simulations, the first plurality of residues in the perturbation set as forming a stable binding between the compound and the first conformation of the protein molecule.

Item 72: The non-transitory computer readable medium of claim 71, wherein the instructions further result in operations comprising the method of any of Items 2 to 35.

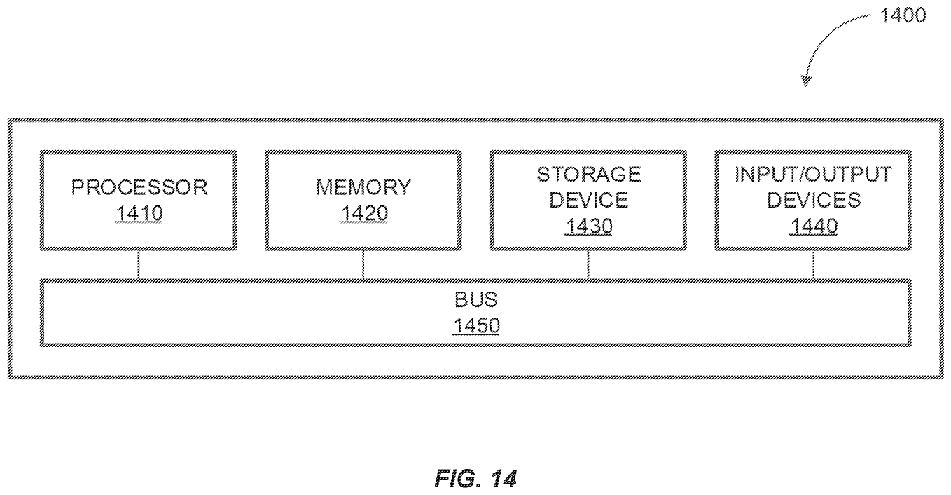

FIG. 14 depicts a block diagram illustrating an example of a computing system 1400, in accordance with some example embodiments. In some example embodiments, the computing system 1400 can be used to implement the analysis controller 110 and/or any components therein. As shown in FIG. 14, the computing system 1400 can include a processor 1410, a memory 1420, a storage device 1430, and input/output devices 1440. The processor 1410, the memory 1420, the storage device 1430, and the input/output devices 1440 can be interconnected via a system bus 1450. The processor 1410 is capable of processing instructions for execution within the computing system. Such executed instructions can implement one or more components of, for example, analysis controller 110 (e.g., Molecular dynamics simulation engine 114, Perturbation set engine 112, etc.). In some example embodiments, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface provided via the input/output device 1440.

The memory 1420 is a computer readable medium such as volatile or non-volatile that stores information within the computing system 1400. The memory 1420 can store data structures representing configuration object databases, for example. The storage device 1430 is capable of providing persistent storage for the computing system 1400. The storage device 1430 can be a floppy disk device, a hard disk device, an optical disk device, a tape device, a solid-state drive, and/or other suitable persistent storage means. The input/output device 1440 provides input/output operations for the computing system 1400. In some example embodiments, the input/output device 1440 includes a keyboard and/or pointing device. In various implementations, the input/output device 1440 includes a display unit for displaying graphical user interfaces.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

As used herein, the conformation of a protein molecule (or a protein conformation) is a three-dimensional arrangement of residues in a sequence of amino acid residues forming the protein molecule. A residue is an organic molecule that includes an alpha carbon linked to an amino group, a carboxyl group, a hydrogen atom, and a variable component called a side chain. As used herein, a coupled protein-structure-compound is a coupling or binding between a protein molecule (having a particular conformation) and a compound (e.g., a ligand). In some cases, the protein molecule and the compound in the coupled protein-structure-compound can be bound to one another (e.g., distance between the center of mass of the compound and the center of mass of the nitrogen atoms in the perturbation set of the protein molecule is smaller than a threshold distance value). In some cases, the protein molecule and the compound in the coupled protein-structure-compound are dissociated or not bound to one another (e.g., distance between the center of mass of the compound and the center of mass of the nitrogen atoms in the perturbation set of the protein molecule is greater than the threshold distance value). As used herein, structural information associated with a particular conformation of a protein molecule includes the spatial arrangement and/or orientation of atoms in each constituent amino acid residue forming the protein molecule (e.g., relative spatial locations and orientation of atoms in the protein molecule).

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. Any recitation herein of the term “comprising”, particularly in a description of components of a composition or in a description of steps of a method, is understood to encompass those compositions and methods consisting essentially of and consisting of the recited components or steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as dis-cussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The subject matter described herein may be embodied in systems, apparatus, methods, and/or articles depending on the desired configuration. For example, apparatuses and/or processes described herein can be implemented using one or more of the following: a processor executing program code, an application-specific integrated circuit (ASIC), a digital signal processor (DSP), an embedded processor, a field programmable gate array (FPGA), and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. These computer programs (also known as programs, software, software applications, applications, components, program code, or code) include machine instructions for a programmable processor and may be implemented in a high-level procedural and/or object-oriented programming language, and/or in assembly/machine language. As used herein, the term “machine-readable medium” refers to any computer program product, computer-readable medium, computer-readable storage medium, apparatus and/or device (e.g., magnetic discs, optical disks, memory, Programmable Logic Devices (PLDs)) used to provide machine instructions and/or data to a programmable processor, including a machine-readable medium that receives machine instructions. Similarly, systems are also described herein that may include a processor and a memory coupled to the processor. The memory may include one or more programs that cause the processor to perform one or more of the operations described herein.

Although a few variations have been described in detail above, other modifications or additions are possible. In particular, further features and/or variations may be provided in addition to those set forth herein. Moreover, the implementations described above may be directed to various combinations and subcombinations of the disclosed features and/or combinations and subcombinations of several further features disclosed above. In addition, the logic flow depicted in the accompanying figures and/or described herein does not require the particular order shown, or sequential order, to achieve desirable results. Other implementations may be within the scope of the following claims. Furthermore, the specific values provided in the foregoing are merely examples and may vary in some implementations.

Although various aspects of the present disclosure are set out in the claims, other aspects of the present disclosure comprise other combinations of features from the described implementations with the features of the claims, and not solely the combinations explicitly set out in the claims.

1. A computer-implemented method, comprising:
(a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound;
(b) performing one or more rounds of molecular dynamics simulations to generate a result indicative of an interaction between the compound and a first plurality of residues in the first perturbation set; and
(c) identifying, based at least on the result of the one or more rounds of molecular dynamics simulations, the first plurality of residues in the perturbation set as forming a stable binding between the compound and the first conformation of the protein molecule.

2. The method of claim 1, wherein the protection factor associated with each residue in the first conformation corresponds to a difference between a first energy of a residue in a bound state and a second energy of the residue in an unbound state, and wherein the one or more residues are identified for inclusion in the first perturbation set based at least on the protection factor of the one or more residues satisfying one or more thresholds.

3. The method of claim 1, wherein each round of molecular dynamics simulation simulates a temporal evolution of a coupled protein-structure-compound including the compound bound to the first conformation of the protein molecule at the first plurality of residues included in the first perturbation set.

4. The method of claim 1, wherein the determining the first perturbation set further comprises excluding, from the first perturbation set, at least a third residue that requires a threshold quantity of reorientations in order to interact with the compound.

5. The method of claim 4, wherein the threshold quantity of reorientations is satisfied by (i) a quantity of atoms in the third residue that require a reorientation in order to interact with the compound, (ii) an angle of reorientation that one or more atoms in the third residue is required to undergo in order to interact with the compound, and/or (iii) a distance of reorientation that the one or more atoms in the third residue is required to undergo in order to interact with the compound.

6. The method of claim 4, further comprising:
determining a first vector from a center of geometry (COG) of the first plurality of residues in the first perturbation set and an α-carbon of the third residue;
determining a second vector from the α-carbon to one of a δ-carbon, γ-carbon, or—carbon present in the third residue; and
determining that the threshold quantity of reorientations is satisfied based at least on an angle formed by the first vector and the second vector satisfying one or more thresholds.

7. The method of claim 1, wherein the determining the first perturbation set further comprises
determining a distance between a first centroid of a first cluster of residues and a second centroid of a second cluster of residues,
determining that the distance fails to correspond to one or more dimensions of the compound, and
excluding, from the first perturbation set, the first residue based at least on the first residue being a part of the first cluster of residues.

8. The method of claim 7, wherein the determining the first perturbation set further comprises
determining a first quantity of residues in the first cluster of residues and a second quantity of residues in the second cluster of residues,
determining a first mean distance between the first centroid and residues in the first cluster,
determining a second mean distance between the second centroid and residues in the second cluster, and
excluding, from the first perturbation set, the first cluster of residues based at least on a determination that (i) the second cluster includes fewer residues than the first cluster, and (ii) the distance between the first centroid of the first cluster and the second centroid of the second cluster exceeds a sum of the first mean distance between the first centroid and residues in the first cluster and the second mean distance between the second centroid and residues in the second cluster.

9. The method of claim 1, wherein the result comprises at least one of
(i) a first distance between a first center of geometry of the compound and a second center of geometry of a plurality of backbone nitrogen (N) atoms in the first plurality of residues in the first perturbation set and (ii) a second distance between a first center of geometry of the compound and one or more amide nitrogen atoms present in the first plurality of residues in the first perturbation set.

10. The method of claim 1, wherein the one or more rounds of molecular dynamics simulation comprise a first round of molecular dynamics simulation performed at a first temperature and a second round of molecular dynamics simulation performed at a second temperature.

11. The method of claim 1, wherein the one or more rounds of molecular dynamics simulation includes a first round of molecular dynamics simulation performed for a first length of time and a second round of molecular dynamics simulation performed for a second length of time.

12. The method of claim 1, wherein the one or more rounds of molecular dynamics simulation includes a first round of molecular dynamics simulation performed for the first conformation of the protein molecule that is associated with the first perturbation set and a second round of molecular dynamics simulation performed for a second conformation of the protein molecule that is associated with a second perturbation set.

13. The method of claim 1, wherein each round of molecular dynamics simulation subjects a coupled compound-protein-structure including the compound bound to the protein molecule at the first plurality of residues in the first perturbation set to a different condition, wherein the result of the one or more rounds of molecular dynamics simulation includes one or more conditions in which the coupled compound-protein-structure disassociates, and wherein the one or more conditions include at least one of (i) a temperature at which the coupled compound-protein-structure dissociates and (ii) a length of time after which the coupled compound protein-structure dissociates.

14. The method of claim 1, wherein the one or more rounds of molecular dynamics simulation includes a round of molecular dynamics simulation that is performed at a higher temperature than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

15. The method of claim 1, wherein the one or more rounds of molecular dynamics simulation includes a round of molecular dynamics simulation that is performed at a lower temperature than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

16. The method of claim 1, wherein the one or more rounds of molecular dynamics simulation includes a round of molecular dynamics simulation that is performed for a longer time period than another round of molecular dynamics simulation in response to a coupled compound-protein-structure including the compound bound to the protein molecule at the first plurality of residues in the first perturbation set failing to dissociate during or at an end of a threshold quantity of other rounds of molecular dynamics simulation.

17. The method of claim 1, wherein the one or more rounds of molecular dynamics simulation includes a round of molecular dynamics simulation that is performed for a shorter time period than another round of molecular dynamics simulation in response to a coupled compound-protein-structure including the compound bound to the protein molecule at the first plurality of residues in the first perturbation set having dissociated during or at an end of a threshold quantity of other rounds of molecular dynamics simulation.

18. The method of claim 1, wherein the first plurality of residues in the perturbation set is identified as forming a stable binding by at least
determining, based at least on the result of the one or more rounds of molecular dynamics simulations, one or more metrics quantifying a binding affinity between the compound and the first conformation of the protein molecule, and
determining, based at least on the one or more metrics, the first plurality of residues in the perturbation set as forming the stable binding between the compound and the first conformation of the protein molecule.

19. A system, comprising:
at least one data processor; and
at least one memory storing instructions, which when executed by the at least one data processor, result in operations comprising;
(a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound;
(b) performing one or more rounds of molecular dynamics simulations to generate a result indicative of an interaction between the compound and a first plurality of residues in the first perturbation set; and
(c) identifying, based at least on the result of the one or more rounds of molecular dynamics simulations, the first plurality of residues in the perturbation set as forming a stable binding between the compound and the first conformation of the protein molecule.

20. A non-transitory computer readable medium storing instructions, which when executed by at least one data processor, result in operations comprising;
(a) determining a first perturbation set for a first conformation of a protein molecule, the determining the first perturbation set comprising identifying, based at least on a protection factor of at least a portion of residues in the protein molecule, one or more residues for inclusion in the first perturbation set, the determining the first perturbation set further includes excluding, from the first perturbation set, at least a first residue whose distance to a second residue in the first perturbation set fails to correspond to one or more dimensions of a compound;
(b) performing one or more rounds of molecular dynamics simulations to generate a result indicative of an interaction between the compound and a first plurality of residues in the first perturbation set; and
(c) identifying, based at least on the result of the one or more rounds of molecular dynamics simulations, the first plurality of residues in the perturbation set as forming a stable binding between the compound and the first conformation of the protein molecule.
